(19) United States
(12) Patent Application Publication
(10) Pub. No.: US 2019/0321330 A1
(43) Pub. Date: Oct. 24, 2019

(54) CANNABIS PLACEBO COMPOSITIONS, DELIVERY VEHICLES AND A METHOD FOR COLOUR MATCHING/NEUTRALIZATION OF CANNABIS PRODUCTS

(71) Applicant: CANOPY GROWTH CORPORATION, Smith Falls (CA)

(72) Inventors: Ben Aron GEILING, Smith Falls (CA); Scott YOUNG, Smith Falls (CA)

(21) Appl. No.: 16/388,222

(22) Filed: Apr. 18, 2019

Related U.S. Application Data
(60) Provisional application No. 62/659,488, filed on Apr. 18, 2018.

(51) Int. Cl. A61K 31/352

(52) U.S. Cl. CPC A61K 31/352

(57) ABSTRACT

Disclosed herein are cannabis placebo compositions having an appearance, odor and flavour to match that of an active cannabis product, as well as methods for colour matching and colour neutralization of cannabis products. The compositions include a colourant to provide a colour match between the cannabis placebo composition and the active cannabis product; and a terpene, an ester, a flavonoid, or any combination thereof to provide an odor and flavour.

### CROSS-REFERENCE TO RELATED APPLICATIONS

This application claims the benefit of and priority to U.S. Provisional Patent Application No. 62/659,488 filed on Apr. 18, 2018, which is incorporated herein by reference in its entirety.

### TECHNICAL FIELD

The present disclosure generally relates to cannabis placebo compositions having an appearance, odor and flavour to match an active cannabis product, as well as methods for colour matching and colour neutralization of cannabis products.

### BACKGROUND

Cannabis is under intense study for use in pharmaceutical, medicinal and recreational fields. Cannabis-derived compositions are under equal scrutiny for use in the same fields. One interesting aspect of cannabis and cannabis-derived compositions is the large number of reputed biological effects. As a very rough segregation, uses involving psychoactive properties may be separated from other uses. Tetrahydrocannabinol (THC) is an example of just one psychoactive cannabinoid that is currently under investigation, whereas cannabidiol (CBD) is an example of a non-psychoactive cannabinoid.

According to some accounts, cannabis is composed of at least 483 known chemical compounds, which include cannabinoids, terpenoids, flavonoids, nitrogenous compounds, amino acids, proteins, glycoproteins, enzymes, sugars and related compounds, hydrocarbons, alcohols, aldehydes, ketones, acids, fatty acids, esters, lactones, steroids, terpenes, non-cannabinoid phenols, vitamins, and pigments. Various cannabinoids have displayed intriguing biological activities, and methods of extracting, refining and purifying certain cannabinoids to high levels of purity and potency exist, such as CBD isolates and/or THC distillates. However, the therapeutic, medicinal and/or recreational benefits of cannabinoids are at an infancy.

As the full potential of cannabinoids for treating diseases and disorders is explored, and as new cannabinoids are selected as candidates for study or new treatment indications identified, active cannabis products will need to undergo placebo-controlled experimentation (e.g. clinical trials) to prove their safety, efficacy and effect. Proper placebo compositions will be needed for testing the effects of both active cannabis products containing a single active compound (e.g. a purified cannabinoid) and active cannabis products containing a combination cannabinoids with active agents. For example, in testing combinations of psychoactive cannabinoids with other active agents (e.g. a non-psychoactive cannabinoid or other drug), placebo compositions with a matching cannabinoid profile of the psychoactive cannabinoid(s) will be required so that the cannabis placebo is indistinguishable from the active cannabis product containing the active agent under study.

A need therefore exists for cannabis placebo compositions that are indistinguishable in colour, odor and flavour from the active cannabis product under study, including a cannabis placebo composition with a matching profile of cannabinoids that are not under study to prove the effect of candidate active agents in cannabis combination products. As well, efficient methods of colour matching and colour neutralization are required to ensure that the cannabis placebo compositions are identical in colour.

### SUMMARY

The present disclosure provides cannabis placebo compositions having an appearance (e.g. colour), odor and flavour to match active cannabis products, as well as methods for colour matching and colour neutralization of cannabis products.

In an aspect, the present disclosure relates to a cannabis placebo composition comprising: (i) a base component matched to that of an active cannabis product; (ii) a colourant to provide a colour match between the cannabis placebo composition and the active cannabis product; and (iii) a terpene, an ester, a flavonoid, or any combination thereof to provide an odor and flavour match between the cannabis placebo composition and the active cannabis product. The cannabis placebo composition as disclosed herein is indistinguishable in appearance, odor and flavour from the active cannabis product.

In an embodiment, the cannabis placebo compositions disclosed herein are devoid of cannabinoids. Placebo compositions of the present disclosure that are devoid of cannabinoids provide a suitable composition to observe the placebo response as compared to an active cannabis product with any number of active ingredients under study, including any and all cannabinoids in the active cannabis products. In an alternative embodiment, the cannabis placebo compositions disclosed herein comprise one or more cannabinoids, such as psychoactive cannabinoids. Placebo compositions of the present disclosure that comprise cannabinoids (i.e. secondary cannabinoids) provide a suitable composition to observe the placebo response as compared to an active cannabis product containing cannabinoids that are not under study and other active agents that are under study (e.g. primary cannabinoids and/or other drugs).

In another aspect, the present disclosure relates to a method for producing a colour-matched placebo, the method comprising: providing an active sample of an active cannabis product; analyzing the active sample by spectrophotometry to obtain one or more absorbance measurements between 380 nm to 780 nm; converting the one or more absorbance measurements into absolute CIE XYZ values; and adding one or more colourants to a cannabis placebo composition to match the absolute CIE XYZ values of the active sample, thereby producing a colour-matched placebo.

In an embodiment, the method for producing a colour-matched placebo further comprises obtaining corresponding absolute CIE XYZ values for a placebo sample of the cannabis placebo composition. This may be performed by providing the placebo sample, analyzing the placebo sample by spectrophotometry to obtain corresponding absorbance measurements between 380 nm to 780 nm (i.e. at the same wavelength as was done for the active sample); and converting the corresponding absorbance measurements into absolute CIE XYZ values.

In an embodiment, the method for producing a colour-matched placebo further comprises determining the difference between the absolute CIE XYZ values of the active sample and the placebo sample.

In an embodiment, the method for producing a colour-matched placebo further comprises repeating the method steps involving analyzing and comparing the absolute CIE XYZ values of the active sample and the placebo sample, and adding one or more colourants to the cannabis placebo composition until the colour-matched placebo is obtained.

In another aspect, the present disclosure relates to a method for neutralizing the colour of a cannabis placebo composition or an active cannabis product, the method comprising: providing a sample of a cannabis placebo composition or an active cannabis product; analyzing the sample by spectrophotometry to obtain one or more absorbance measurements between 380 nm to 780 nm; converting the one or more absorbance measurements into absolute CIE XYZ values; and adding a colourant that is opposite to an undesired colour to reduce the effect of the undesired colour, thereby neutralizing the colour of the cannabis placebo composition or an active cannabis product.

Other aspects and features of the cannabis placebo compositions and methods of the present disclosure will become apparent to those ordinarily skilled in the art upon review of the following description of specific embodiments.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 shows the colour matching of a cannabis placebo composition in accordance with the methods of the present disclosure. Left to Right: (i) Cannabis placebo oil having no cannabinoids, and active cannabis products (ii) Spectrum Red cannabis oil, (iii) Argyle cannabis oil and (iv) Spectrum Yellow cannabis oil. Although it cannot be observed in the black-and-white rendering, the compositions had a near identical honey colour.

FIG. 2 shows CIE 1931 XYZ color space diagrams.

FIG. 3 is a colour wheel showing opposite colours. Opposite colours are capable of neutralizing effects of colours.

### DETAILED DESCRIPTION

The present disclosure provides cannabis placebo compositions, using naturally derived components and a repeatable process. The cannabis placebo compositions disclosed herein are indistinguishable in appearance (e.g. colour), odor and flavour as compared to active cannabis products. The cannabis placebo compositions disclosed herein may be ideal for use in double-blinded, placebo controlled clinical trials.

Regardless of the mode of administration (e.g. oil, capsule or tablet), the cannabis placebo compositions are indistinguishable from counterpart active cannabis products. Moreover, using the same repeatable process and naturally derived ingredients, the colour, odor and flavour of cannabis placebo compositions (e.g. oil) can be replicated to produce a product that is identical to an active cannabis product (e.g. cannabis oil). The methods of colour matching of the present disclosure can likewise be used to neutralize the appearance of undesired colours in a variety of cannabis products.

In accordance with the methods disclosed herein, cannabis placebo compositions may be prepared that mitigate variance between the placebo and the active cannabis products by reducing the effect of human perception. The cannabis placebo compositions are colour matched to the active cannabis product using a sophisticated process. This has great advantage over trial and error colour-matching techniques, such as titration, which would be time-consuming and would produce inconsistent results due to differences in perception of organoleptic properties.

Moreover, in advantageous aspects, the use of naturally derived components provide a cannabis placebo composition that is generally considered to be healthier than products that contain synthetic ingredients. In this regard, the methods herein provide a means for calculating the dye effect values (i.e. colour changing properties) on CIE XYZ values.

In an embodiment, the cannabis placebo compositions disclosed herein are devoid of cannabinoids. By “devoid of cannabinoids”, it is meant that the cannabis placebo compositions are free of cannabinoids. These types of placebo compositions are advantageous for studying the effect of one or more cannabinoids in an active cannabis product. From the perspective of the subject, the cannabis placebo composition will be indistinguishable from the active cannabis product in terms of appearance (e.g. colour), odor and flavour.

In addition to being a placebo, the above cannabis placebo composition that is devoid of cannabinoids may provide a particularly suitable cannabis delivery vehicle, thereby acting as a system for delivery of one or more active agents to a subject. Thus, a delivery vehicle or delivery system is itself contemplated as falling within the scope of the present disclosure. The cannabis delivery vehicle may include one or more cannabinoids and/or one or more other active agents. The subject in need of the active agent(s) will experience consumption of the active agent(s) as indistinguishable from the consumption of cannabis or a cannabis-derived composition.

In another embodiment, the cannabis placebo compositions disclosed herein may comprise one or more cannabinoids (“secondary cannabinoids”). These types of placebo compositions are advantageous for studying the effect of active agents in combination with cannabinoids. The active agent may itself be a cannabinoid (“primary cannabinoid”) or may by a different active ingredient, such as a drug. In particularly advantageous embodiments, the cannabinoids in the cannabis placebo composition are psychoactive cannabinoids and thereby render the placebo indistinguishable from an active cannabis product that contains psychoactive cannabinoids in combination with other active agents under study.

These and other objects, features, and advantages of the present disclosure will be apparent to those skilled in the art from the following detailed description of the embodiments.

### Cannabis

Cannabis is a genus of flowering plant in the family Cannabaceae. The number of species within the genus is disputed. Three species may be recognized, Cannabis sativa, Cannabis indica and Cannabis ruderalis. C. ruderalis may be included within C. sativa; or all three may be treated as subspecies of a single species, C. sativa. (See e.g. G. W. Guy; B. A. Whittle and P. Robson (Eds.), “The Medicinal Uses of Cannabis and Cannabinoids”, Pharmaceutical Press., pp. 74, 2004, ISBN 978-0-85369-517-2; M. Colbert, “Indica, Sativa, Ruderalis—Did We Get It All Wrong?”, The Leaf Online, Jan. 26, 2015.)

Cannabis sativa is an annual herbaceous plant in the Cannabis genus. It is a member of a small, but diverse family of flowering plants of the Cannabaceae family. It has been cultivated throughout recorded history, used as a source of industrial fiber, seed oil, food, recreation, religious and spiritual moods and medicine. Each part of the plant is harvested differently, depending on the purpose of its use. The species was first classified by Carl Linnaeus in 1753.

Cannabis indica, formally known as Cannabis sativa forma indica, is an annual plant in the Cannabaceae family. A putative species of the genus Cannabis.

Cannabis ruderalis is a low-THC species of Cannabis that is native to Central and Eastern Europe and Russia. It is widely debated as to whether C. ruderalis is a sub-species of Cannabis sativa. Many scholars accept Cannabis ruderalis as its own species due to its unique traits and phenotypes that distinguish it from Cannabis indica and Cannabis sativa.

The genus Cannabis is indigenous to central Asia and the Indian subcontinent (M. A. ElSohly (Ed.), “Marijuana and the Cannabinoids”, Humana Press. p. 8, 2007, ISBN 1-58829-456-0). Cannabis has long been used for hemp fiber, for hemp oils, for medicinal purposes, and as a recreational drug. Industrial hemp products are made from cannabis plants selected to produce an abundance of fiber. To satisfy the UN Narcotics Convention, some cannabis strains have been bred to produce minimal levels of tetrahydrocannabinol (THC), the principal psychoactive constituent. Many additional plants have been selectively bred to produce a maximum of THC (cannabinoids), which is obtained by curing the flowers. Various compounds, including hashish and hash oil, may be extracted from the plant.

Within naturally occurring and manmade hybrids, cannabis contains a vast array of compounds. Four compound classes are of particular interest within the context of the present disclosure, although other compounds can be present and/or added. These four classes include cannabinoids, terpenes, esters and flavonoids.

There are many ways of growing cannabis some of which are natural, and some of which are carefully designed by humans and they will not be recited here. However, one of ordinary skill in the art of cannabis production will typically place a cannabis seed or cutting into a growth media such as soil, manufactured soil designed for cannabis growth, or one of many hydroponic growth mediums. The cannabis seed or cutting is then provided with water, light and, optionally, a nutrient supplement. At times, the atmosphere and temperature are manipulated to aid in the growth process. Typically, the humidity, air to carbon dioxide gas ratio and elevated temperature, either by use of a heat source or waste heat produced by artificial light are used. On many occasions, ventilation is carefully controlled to maintain the conditions described above within an optimal range to both increase the rate of growth and, optionally, maximize the plant's production of, for example, cannabinoids. It is possible to control lighting cycles to optimize various growth parameters of the plant.

Given the number of variables and the complex interaction of the variables, it is possible to develop highly specific formulas for production of cannabis that lead to a variety of desired plant characteristics. The present disclosure, particularly in respect of active cannabis products to which a placebo of the present disclosure may be compared, is applicable to use with such means for growing cannabis as well as any of the variety of conventional and modified methods.

### Placebo

In general terms, a placebo is a substance, composition or treatment of no intended therapeutic value. In drug testing and medical research, a placebo is made to resemble an active medication so that it functions as a control. In a single-blind clinical trial, only the investigators would know which treatment a subject received (i.e. placebo or active). In a double-blind clinical trial, both the investigators and the subject are prevented from knowing which treatment the subject received.

Placebos are therefore used to provide test data on treatment subjects wherein the subjects cannot distinguish between the placebo and an active product under study. Such data are necessary to determine the efficacy of a candidate active agent as compared to the so-called placebo effect.

The placebo effect is part of the response to any active medical intervention. The placebo effect demonstrates the importance of perception and the brain's role in physical health. Psychological responses to some placebos are more effective than others. Large pills seem to work better than small pills, colored pills work better than white pills, an injection is more powerful than a pill, and so on. Other important factors in the effect of placebos include smell, odor, taste, organoleptic, texture and any subject observable similarity to known active drugs. The “placebo effect” is determined by comparing the placebo response to any response observed in subjects that received no treatment at all.

When selecting the composition of a placebo for use in scientific or clinical use, it is important that the intended subject is unable to distinguish the placebo from the active product under study. Thus, by its nature, a placebo is indistinguishable from an active product under study by a human or other animal subject.

### Cannabis Placebo Composition

The present disclosure provides cannabis placebo compositions that are indistinguishable in appearance (e.g. colour), odor and flavour as compared to corresponding active cannabis products.

By “corresponding”, it is meant to refer to placebo and active products of the same dosage form (e.g. oil, gel capsule, tablet, etc), whereby the cannabis placebo composition of the present disclosure is intended to be a placebo to that specific “corresponding” active cannabis product. The terms “corresponding” and “counterpart” may be used interchangeably herein.

Within the context of the present disclosure, the term “indistinguishable” has the common English language meaning. In particular, the term means that a consumer (e.g., a clinical trial subject) is unable to tell the difference between an active cannabis product and a cannabis placebo composition of the present disclosure that is designed to be a placebo to that active cannabis product. Thus, the subject is not able to distinguish between a test composition containing an active agent under study (i.e. active cannabis product) and a corresponding cannabis placebo composition of the present disclosure that does not contain that active agent.

In an embodiment, indistinguishable is a subjective measure in that individual subjects themselves are not able to tell the difference. If by human perception a cannabis placebo composition of the invention cannot be identified as different than a corresponding active cannabis product, then the products are considered indistinguishable. In another embodiment, indistinguishable is an objective measure such as by scientific observations, calculations and/or determinations in respect of the colour, odor and flavour of the cannabis placebo composition as compared to the active cannabis product. In an embodiment, the scientific measurements of colour, odor and flavour do not deviate by more than 10%, 9%, 8%, 7%, 6%, 5%, 4%, 3%, 2%, 1%, 0.5%, 0.25% or 0.1% as between the cannabis placebo composition and the active cannabis product.

As used herein, the term “subject” refers to an animal. In an embodiment, the animal is a human, a companion animal or livestock. In an embodiment, the companion animal is a dog. In an embodiment, the subject is a human involved in a clinical trial. In an embodiment, the subject is in need of treatment with an active agent (e.g. a cannabinoid or a drug). In an embodiment, the subject is a user intending to experience cannabis or a cannabis-derived composition together with one or more other active agents.

As used herein, the term “active cannabis product” refers to cannabis or a cannabis-derived composition that comprises at least one active agent that is not present in the corresponding cannabis placebo composition. In an embodiment, the active agent is a single cannabinoid that is under study. In an embodiment, the active agent is a combination of cannabinoids that are under study. The combination of cannabinoids may include 2, 3, 4, 5, 6, 7, 8, 9, 10 or more cannabinoids. Indeed, it is contemplated herein that not all of the cannabinoids in the active cannabis product will be known. For example, active cannabis products may be prepared by extraction methods (e.g. solvent and/or mechanical extraction) and all of the various different cannabinoids and their amounts may not be known. However, using consistent plants (i.e. of the same strain and growth conditions) and identical extraction methods, it may be possible to repeatedly and reproducibly prepare substantially identical active cannabis products for therapeutic, medicinal and/or recreational purposes.

The term “primary cannabinoid” is used herein to refer to the one or more cannabinoids that are present in the active cannabis product, but are not present in the corresponding cannabis placebo composition of the present disclosure. As such, “primary cannabinoid” refers to the one or more cannabinoids that are under study to determine their effect as compared to the placebo effect. In an embodiment, the primary cannabinoids may be non-psychoactive cannabinoids.

A “primary cannabinoid” is to be distinguished herein from a “secondary cannabinoid”. The term “secondary cannabinoid”, as used herein, refers to the one or more cannabinoids that are present in both the cannabis placebo composition and the active cannabis product. As such, “secondary cannabinoid” does not refer to the one or more cannabinoids that are under study against the placebo. In an embodiment, the secondary cannabinoids may be psychoactive cannabinoids present in the active cannabis product, to which the psychoactive effect is matched in the cannabis placebo composition to provide a placebo that is indistinguishable from the active cannabis product in respect of that psychoactive effect.

In another embodiment, the active agent in the active cannabis product is a compound or substance other than a cannabinoid that is capable of generating an effect in a subject. In an embodiment, the active agent may be a terpene. In an embodiment, the active agent may be a drug. As used herein the term “drug” has its common meaning as any substance that causes a physiological and/or psychological change in the body. In an embodiment, the drug is a pharmaceutical drug that may be used to treat, cure or prevent a disease or disorder, ameliorate one or more symptoms of a disease or disorder, or generally promote a better well-being in a subject.

In addition or in alternative to the active cannabis product comprising one or more cannabinoids, the active cannabis product may comprise a drug. In an embodiment, the active cannabis product comprises as an active agent only a drug other than a cannabinoid. More preferably, the active cannabis product comprises a drug in combination with either one or more primary cannabinoids, one or more secondary cannabinoids, or any combination thereof. In a particular embodiment, the active cannabis product comprises a drug in combination with one or more secondary cannabinoids.

The active cannabis product may be produced by any suitable means. In an embodiment, the active cannabis product is produced by using the cannabis placebo composition herein as a delivery vehicle and adding the active agent(s) to be studied. In such embodiments, the present disclosure further provides a system for delivery of one or more active agents to a subject. The delivery system itself is contemplated as falling within the scope of the present disclosure.

More preferably, the active cannabis product is a cannabis-derived composition. As used herein, a cannabis-derived composition refers to any composition that is derived from a cannabis plant. By “derived from”, it is meant that the active cannabis product, or a portion thereof, is obtained from a cannabis plant source. The active cannabis product need not be entirely obtained from a cannabis plant, but rather at least a portion of the material is sourced from a cannabis plant. Moreover, the material may have been subsequently modified, such as by preparing the cannabis-derived material in a dosage form as described herein (e.g. an oil, a gel capsule, a tablet, a cream, etc.), extracting, purifying or removing one or more components (e.g. cannabinoids, terpenes, plant waxes, etc.), or adding one or more components (e.g. other active agents, carriers, excipients, etc.).

Methods of converting cannabis plant matter into a cannabis-derived composition typically involves harvesting and, optionally, one or more steps of extraction, fractionation, or purification. More typically, a combination of two or more such steps are employed. More typically still, a combination of separating the cannabis from the media in which it is grown, drying to reduce the water content, grinding to form a power, extraction and, optionally, a fractionation or purification step is performed.

In an embodiment, the cannabis is separated from the media in which it is grown and first dried and then ground. Once in the ground state it is, optionally, sieved and finally the resins of the plant are extracted. These resins are an embodiment of an active cannabis product of the disclosure, and additional synthetic, semisynthetic or naturally derived compounds may be added to the resins. Remembering that optional fractionation and purification steps are possible, embodiments of the active cannabis products of the disclosure may have compounds removed from the resin. At that point, again optionally, synthetic, semisynthetic or naturally derived compounds may be added to the resin to form embodiments of the active cannabis products of the disclosure.

In a particular embodiment, the cannabis-derived composition is a cannabis extract, a cannabis concentrate or a composition containing the extract or concentrate. Various types of extracts and concentrates are well known to the skilled person, as well as methods to obtain extracts and concentrates.

In an embodiment, the cannabis-derived composition is a cannabis oil. Various cannabis oil compositions and methods for their preparation are described, for example, in WO 2017/091764. Thus, in an embodiment, the active cannabis product is an oil. In an embodiment, the oil may be encapsulated to form a gel capsule. Thus, in an embodiment the active cannabis product is a gel capsule.

The present disclosure provides cannabis placebo compositions that are indistinguishable in appearance (e.g. colour), odor and flavour to corresponding active cannabis products.

In an embodiment, the present disclosure relates to a cannabis placebo composition comprising: (i) a base component matched to that of an active cannabis product; (ii) a colourant to provide a colour match between the cannabis placebo composition and the active cannabis product; and (iii) a terpene, an ester, a flavonoid, or any combination thereof to provide an odor and flavour match between the cannabis placebo composition and the active cannabis product, wherein the cannabis placebo composition is indistinguishable in appearance, odor and flavour from the active cannabis product.

As used herein, the term “base component” describes the one or more ingredients that provide the dosage form of the cannabis placebo composition. The base component may include, for example and without limitation, carriers, excipients and other additives. Exemplary references describing dosage forms include: “Remington: The Science and Practice of Pharmacy”, 21st ed., Publisher: Wolters Kluwer, May 19, 2005, ISBN-13 9780781746731, as well as “Ansel's: Pharmaceutical Dosage Forms and Drug Delivery Systems”, 10th ed., Publisher: Wolters Kluwer, Dec. 31, 2013, ISBN-13: 978-1451188769.

In respect of the cannabis placebo compositions, exemplary dosage forms include, without limitation, an oil, a gel capsule, a tablet, a lozenge or pastille, a pill, a granule, a powder, a cream, a suppository, an enema, or a liquid preparation. In an embodiment, a liquid preparation may be a beverage. One of ordinary skill in the pharmaceutical and pharmacological arts is are aware of a wide variety of dosage forms and the constituents used to prepare such dosage forms. In an embodiment, the base components are pharmaceutically acceptable carriers, excipients and additives. In an embodiment, the base components are recreationally acceptable carriers, excipients and additives.

By “matched to”, it is intended to mean that the base component of the cannabis placebo compositions is substantially the same as that of the active cannabis product. For example, if the active cannabis product is an oil, the base component of the cannabis placebo composition is likewise an oil. By “substantially the same”, it is meant that the base components are indistinguishable from one another at the outset or are made to be indistinguishable from one another by the addition of colourants, terpenes, esters, flavonoids, or any combination thereof, in accordance with the present disclosure. In an embodiment, the base component of the cannabis placebo composition is matched to that of an active cannabis product because the base components are identical as between them.

In a particular embodiment, the cannabis placebo composition of the present disclosure is an oil and the base component is a carrier oil. In an embodiment, the carrier oil may be any essential oil or the like. Various exemplary carrier oils and essential oils are described, for example, in WO 2017/091764 and any of these oils may be used in the cannabis placebo compositions of the present disclosure.

In an embodiment, the carrier oil may be medium chain triglyceride (MCT) oil, long chain triglyceride (LCT) oil, coconut oil, corn oil, canola oil, olive oil, avocado oil, vegetable oil, flaxseed oil, palm oil, peppermint oil, hemp oil, sesame oil, sunflower oil, a winterized oil of long-chain mono-, di-, and tri-glycerides (e.g. Maisine® CC), rice bran oil, or any combination thereof.

In an embodiment, the carrier oil is MCT. MCT may represent an advantageous carrier oil for the cannabis placebo composition of the present disclosure because it is nearly colourless, odorless and flavourless.

A carrier oil may be present in the cannabis placebo composition in an amount ranging from about 0.5% to about 99.5%. In an embodiment, a carrier oil may be present in the cannabis placebo composition in an amount of about 5% (w/w), about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w), about 80% (w/w), about 85% (w/w), about 90% (w/w), about 95% (w/w), about 96% (w/w), about 97% (w/w), about 98% (w/w), about 99% (w/w), or more. In an embodiment, a carrier oil may be present in the cannabis placebo composition in an amount ranging from about 90% to about 99.5%. In an embodiment, a carrier oil may be present in the cannabis placebo composition in an amount ranging from about 95% to about 99.5%.

In a particular embodiment, the cannabis placebo composition of the present disclosure can take the form of, e.g., a tablet or a capsule prepared by conventional means with a pharmaceutically acceptable excipient. Tablets and gelatin capsules may comprise any number of base components, including: diluents or fillers, lubricants, binders, disintegrants, and wetting agents.

Exemplary embodiments of diluents and fillers include, without limitation, lactose, dextrose, sucrose, mannitol, maltodextrin, lecithin, agarose, xanthan gum, guar gum, glycerol, sorbitol, cellulose (e.g., ethyl cellulose, microcrystalline cellulose), glycine, pectin, polyacrylates and/or calcium hydrogen phosphate and calcium sulfate.

Exemplary embodiments of lubricants include, without limitation, silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol.

Exemplary embodiments of binders include, without limitation, magnesium aluminum silicate, starch paste, gelatin, tragacanth, methylcellulose, sodium carboxymethylcellulose, polyvinylpyrrolidone and/or hydroxypropyl methylcellulose.

Exemplary embodiments of disintegrants include, without limitation, starches (e.g., potato starch or sodium starch), glycolate, agar, alginic acid or its sodium or potassium salt, or effervescent mixtures.

Exemplary embodiments of wetting agents include, without limitation, sodium lauryl sulfate.

The excipients described herein can also be used for preparation of buccal dosage forms and sublingual dosage forms (e.g., lozenges), for example, in U.S. Pat. Nos. 5,981,552 and 8,475,832. Formulation as creams is also contemplated and the ingredients above may be used, as well as solidifying agents such as shea butter, beeswax, kokum butter, mango butter, ilipe butter, tamanu butter, emulsifying wax, soy wax, castor wax, rice bran wax, and candelila wax.

Cannabinoids, terpenes and flavonoids tend to be lipophilic and typically have low solubility in hydrophilic biocompatible matrix materials. One method for obtaining desirable dosage forms comprising lipophilic substances and hydrophilic biocompatible matrix substances is to encapsulate or disperse lipophilic substances in the hydrophilic matrix using additives or modifiers which provide an environment for stable oil-in-water emulsions, micelles, liposomes or other complex phase equilibrium modified compositions. Exemplary techniques, modifiers and additives are described herein. Such techniques may, for example, be used to prepare liquid preparations of cannabinoids (e.g. beverages) as a cannabis placebo composition of the invention and a corresponding active cannabis product.

An exemplary method of preparing a stable oil-in-water dosage form is to use a nanoemulsion to encapsulate lipophilic bioactive compounds in a carrier oil. The carrier oil is, optionally, food grade, does not adversely affect product quality (such as appearance, taste, texture, or stability), protects the active agents from chemical degradation during storage and distribution, and increases bioavailability following ingestion. Carrier oils help stabilize emulsions from Ostwald ripening, a destabilization mechanism of nanoemulsions. This problem arises due to the increased solubility of the dispersed phase in a hydrophilic medium.

Carrier oils can have an effect on the physicochemical stability of nanoemulsions in the gastrointestinal tract (GI Tract). The rate and extent of lipid digestion is higher for MCT emulsions than for LCT emulsions, which is attributed to differences in the water dispersibility of the medium and long chain fatty acids formed during lipolysis. The total bioavailability of active components after digestion can be higher for LCT emulsions than for MCT emulsions.

LCT contain fatty acids of 12-20 carbon atoms and can form mixed micelles with a hydrophobic core large enough to accommodate active substances such as THC and other cannabinoids, terpenoids and flavonoids. MCT contain fatty acids of 6-12 carbon atoms and can form mixed micelles with smaller hydrophobic cores.

Emulsions can be prepared in concentrated form and later diluted several hundred times in sugar/acid solutions prior to consumption to produce finished dosage forms in either carbonated or non-carbonated biocompatible matrix systems. Selection of an emulsifier can affect the shelf-life and physicochemical properties of the emulsion. Emulsions stabilized by surfactants or other types of stabilizing compounds such as phospholipids, amphiphilic proteins, or polysaccharides, have been developed to provide controlled release, improved entrapment efficiency, and protection from degradation.

Emulsions can be prepared in several ways such as by mechanical processes which employ shear force to break large emulsion droplets into smaller ones, high-pressure homogenization (HPH, including microfluidization) and high-amplitude ultrasonic processing, and ultrasound-assisted emulsification.

Small droplet sizes tend to lead to transparent emulsions. In this regard, droplet sizes of about 100, 90, 80, 70, 60, 50 or 40 nm are desirable. Suitably the droplet sizes for transparent emulsions are in the range of 40 to 60 nm, more suitably they are in the range of 45 to 55 nm, more suitably yet, 50 nm.

Other suitable types of modifiers and additives that may be included in the cannabis placebo compositions disclosed herein to match an active cannabis product include, without limitation, natural emulsifiers, thickening agents, minerals, acids, bases, vitamins, and other processing, storage, distribution, transport, and use conditions such as ultrasonication, nitrogen dosing, packaging, and sterilization.

Nutritional additives and modifiers useful to the consumer may also be included in the cannabis placebo compositions of the present disclose to match the active cannabis product, assuming it is not the nutritional additive that is under study in which case it would not be included in the placebo. Nutritional additives function in the maintenance of normal body health. Suitably nutritional additives comprise essential nutrients including vitamins, dietary minerals amino acids and fatty acids, including without limitation vitamin A, vitamin B1, vitamin B2, vitamin B3, vitamin B5, vitamin B6, vitamin B7, vitamin B9, vitamin B12, vitamin C, vitamin D, vitamin E, vitamin K calcium, phosphorus, potassium, sulfur, sodium, chlorine, magnesium, iron, cobalt, copper, zinc, molybdenum, iodine, selenium, manganese, nickel, chromium, fluorine, boron, strontium histidine, isoleucine, leucine, lysine, methionine, cysteine, phenylalanine, tyrosine, threonine, tryptophan, valine, alpha-linoleic acid, and linoleic acid.

Depending on the dosage form, optional additives and modifiers further comprise one or more of acids, bases, acidity regulators, alcohol, anticaking agents, antifoaming agents, antioxidants, bulking agents, coagulation agents, colour retention agents, emulsifiers, flavor enhancers, flour treatment agents, gelling agents, glazing agents, humectants, leavening agents, tracer gases, preservatives, stabilizers, sweeteners, tenderizers, and thickeners.

One class of common additive or modifier useful in oral dosage forms of the present disclosure is the group of substances referred to as phospholipids. Phospholipids are made up of two fatty acid tails and a phosphate group head. Fatty acids are long chains mostly made up of hydrogen and carbon, while phosphate groups consist of a phosphorus molecule with four oxygen molecules attached. These two components of the phospholipid are connected via a third molecule, glycerol.

Phospholipids can act as emulsifiers, enabling oils to form a colloid with water. Phospholipids are one of the components of lecithin which is found in egg-yolks, as well as being extracted from soy beans, and is used as a food additive in many products and can be purchased as a dietary supplement. Lysolecithins are typically used for water-oil emulsions like margarine, due to their higher HLB ratio.

Dosage forms of this type commonly use phospholipid additives or modifiers to solubilize one or more hydrophobic components of the cannabis or cannabis-derived composition. In this embodiment, phospholipids are typically derived from natural sources such as a naturally occurring oils from a plant such as coconut, safflower and sunflower. These phospholipids can include secondary products obtained therefrom, such as lecithin from sunflower oil. In these embodiments, the phospholipid or derivative thereof is typically present in about 0.01-10% w/w or w/v. More typically, 0.01%, 0.1%, 1% or 10% w/w or w/v, more typically still 0.1% to 1% w/w or w/v.

In the same fashion as phospholipids, triglycerides are a typical additive or modifier of oral dosage forms. Triglycerides are chemically tri-esters of fatty acids and glycerol. Triglycerides are formed by combining glycerol with three fatty acid molecules. Alcohols have a hydroxyl (—OH) group. Organic acids have a carboxyl (—COOH) group. Alcohols and organic acids join to form esters. The glycerol molecule has three hydroxyl (—OH) groups. Each fatty acid has a carboxyl group (—COOH). In triglycerides, the hydroxyl groups of the glycerol join the carboxyl groups of the fatty acid to form ester bonds:

HOCH₂CH(OH)CH₂OH+RCO₂H+R′CO₂H+R″CO₂H→RCO₂CH₂CH(O₂CR′)CH₂CO₂R″+3H₂O

The three fatty acids (RCO₂H, R′CO₂H, R″CO₂H in the above equation) are usually different, but many kinds of triglycerides are known. The chain lengths of the fatty acids in naturally occurring triglycerides vary, but most contain 16, 18, or 20 carbon atoms. Natural fatty acids found in plants and animals are typically composed of only even numbers of carbon atoms, reflecting the pathway for their biosynthesis from the two-carbon building-block acetyl CoA. Bacteria, however, possess the ability to synthesize odd- and branched-chain fatty acids. As a result, ruminant animal fat contains odd-numbered fatty acids, such as 15, due to the action of bacteria in the rumen. Many fatty acids are unsaturated, some are polyunsaturated (e.g., those derived from linoleic acid).

Most natural fats contain a complex mixture of individual triglycerides. Because of this, they melt over a broad range of temperatures. Cocoa butter is unusual in that it is composed of only a few triglycerides, derived from palmitic, oleic, and stearic acids in the 1-, 2-, and 3-positions of glycerol, respectively.

Natural phospholipid derivatives include egg PC (Egg lecithin), egg PG, soy PC, hydrogenated soy PC, and sphingomyelin as natural phospholipids. Synthetic phospholipid derivatives include phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), phosphatidylserine (DOPS), PEG phospholipid (mPEG-phospholipid, polyglycerin-phospholipid, functionalized-phospholipid, and terminal activated-phospholipid).

Phospholipids can form cell, micelle and liposomal membranes as well as other self-organizing multi-molecular structures because the phosphate group head is hydrophilic (water-loving) while the fatty acid tails are hydrophobic (water-hating). They automatically arrange themselves in a certain pattern in water or other polar environment because of these properties, and form membranes. To form membranes, phospholipids line up next to each other with their heads on the outside of the polar medium and their tails on the inside, thus forming an inner and outer surface. A second layer of phospholipids also forms with heads facing the inside of the structure and tails facing away. In this way, a double layer is formed with phosphate group heads on the outside, and fatty acid tails on the inside. This double layer, called a lipid bilayer, forms the main part of the membrane or other similar structure.

Any of the above base components (including additives and modifiers), as well as the described techniques, may be used in the preparation of a cannabis placebo composition of the present disclosure in order to provide a placebo that is a matching dosage form to the active cannabis product. In an embodiment, the base components of the cannabis placebo composition are identical to those of the active cannabis product. In an embodiment, different base components may be used for the cannabis placebo composition than the active cannabis product, whereby the cannabis placebo composition is made indistinguishable from the active cannabis product by the addition of colourants, terpenes, esters, flavonoids, or any combination thereof, in accordance with the present disclosure.

As with dosage forms, countless methods of delivery exist for the active cannabis product and the corresponding cannabis placebo composition of the present disclosure. Within the context of the active cannabis product, delivery includes the provision and use of a dosage form containing the active agent(s) such that about 10%, 20%, 30%, 40%, 50%, 60%, 70%, or 80%, more suitably, about 90%, and more suitably yet, about 95% of the active agent(s) reaches a biological system of the subject such that it is available for biological interaction with said subject. In an aspect of the present disclosure, the cannabis placebo composition disclosed herein can assist in identifying and determining appropriate dosage forms for effective delivery of the active agents.

Suitable amounts of the active agent(s) for use in the dosage forms of the active cannabis product will depend upon many factors including, for example, age and weight of an individual, specific active agent(s) to be used, nature of a composition, whether the composition is intended for direct administration or is a concentrate, and combinations thereof. Ultimately, a suitable dosage can be readily determined by one skilled in the art. For example, one skilled in the art can begin with a low amount that can be increased until reaching the desired result or effect. Alternatively, one skilled in the art can begin with a high dosage that can be decreased until reaching a minimum dosage needed to achieve the desired result or effect. In an aspect of the present disclosure, the cannabis placebo composition disclosed herein can assist in identifying and determining appropriate dosages of the active agents.

The cannabis placebo compositions as disclosed herein comprise one or more colourants. As used herein, the term “colourant” refers to any substance or compound that may be used to provide colour to the cannabis placebo compositions.

In an embodiment, the colourant is a synthetically produced compound. Synthetic colorants may include, but are not limited to, Tartrazine, Quinoline Yellow, Sunset Yellow, Azorubine, Ponceau 4R, Erythrosine, Allura Red, Patent Blue, Indigo Carmine, Brilliant Blue FCF, Green S, Brilliant Black and Brown HT.

More preferably, the colourant is a naturally derived substance or compound. In an embodiment, the naturally derived colourant is derived from an extract or oil from a plant other than cannabis. In this context, by “derived from”, it is meant that the substance or compound is obtained from the plant. It may still be contained together with other plant matter or it may be substantially purified from the plant matter. By “substantially purified”, it is meant that the substance or compound is at least 75% pure (w/w), at least 80% pure (w/w), at least 85% pure (w/w), at least 90% pure (w/w), at least 91% pure (w/w), at least 92% pure (w/w), at least 93% pure (w/w), at least 94% pure (w/w), at least 95% pure (w/w), at least 96% pure (w/w), at least 97% pure (w/w), at least 98% pure (w/w), at least 99% pure (w/w), or more.

In an embodiment, the colourant is derived from Acai berries, Alfalfa, Alkanna tinctoia root, Bixa orellana seed, Persea gratissima oil, Beta vulgaris, Tanacetum annuum flower, Mauritia flexuosa fruit, Clitoria ternatea flower, Calendula officinalis flower, Daucus carota sativa root, Matricaria recutita flower oil, Reseda luteola, Sambucus nigra, Lawsonia inermis, Hibiscus rosa-sinensis flower, Indigofera tinctoria, Iris germanica, Rubia tinctorum, Monascus, Urtica dioica leaf, Capsicum anuum, Punica granatum fruit, Brassica oleracea leaf, Trifolium pretense, Pterocarpus santalinum, Rosa canina fruit oil, Carthamus tinctorius seed oil, Crocus sativus, Hippophae rhamnoides fruit oil, Spinacia oleracea leaf, Spirulina platensis, Hypericum perforatum flower, Solanum lycopersicum, Curcuma longa, Juglans nigra shell, Isatis tinctoria leaf, Achillea millefolium oil, or any combination thereof.

In an embodiment, the colourant comprises or consists of anthocyanins, e.g. cyaniding-3-rutinoside and cyanidin-3-glucoside (red/purple/blue), alkannin (red/purple), alizarin (red/purple), beetroot (red/pink), Beta-carotene (orange), betanin (red/pink), capsanthin (red/purple), capsorubin (red/purple), caramel (amber/brown), carbon black (black), carmine (deep-red), carotenoids, e.g. norbixin, bixin (orange/yellow/red), carthamin (yellow/red), chamazulene (blue), chlorophyll (green), curcumin (yellow/orange), crocin (yellow), crocetin (yellow), cyanidin-3-sophoroside (red/pink), cyanidin-3-sambubioside (red/pink), delphinidin-3-sambubioside (red/pink), delphinidin (purple/blue), flavoxanthin (orange), formononetin (golden yellow), hypericin (red), indigotin (blue/mauve), juglone (orange/brown), lawsone (red/orange), lutein (green/yellow), luteolin (yellow), lycopene (red/orange), mangiferin (purple/blue/green), paprika (red-orange), phycocyanin (blue/green), phycoerthyrin (blue/green), picrocrocin (yellow), punicalagin (red/purple), purpurin (red/purple), riboflavin (yellow-orange), rubropunctamine (red/purple), santalin (red), titanium dioxide (white), zeaxanthin (orange), or any combination thereof.

In a particular embodiment, the colourant comprises anthocyanins, beetroot, caramel, carbo black, carmine, carotenoids, chlorophyll, curcumin, paprika, riboflavin, titanium dioxide, or any combination thereof.

In a particular embodiment, the colourant is comprised of a beta-carotene extract, an alfalfa extract, or any combination thereof.

Depending on the cannabis placebo composition to be prepared, the colourant may be a single colourant or may be any mixture of colourants to provide the desired change in colour to a non-colour-matched placebo. The individual colourants may be added to the cannabis placebo composition one at a time, may be added to the cannabis placebo composition as a mixture, or any combination thereof, to provide colour matching to the active cannabis product.

The present disclosure provides advantageous methods to produce a colour-matched cannabis placebo composition. In an embodiment, this is achieved by experimentally calculating the dye effect on CIE XYZ values, as described herein. By “dye effect”, as used herein, it is meant the ability of the colourant to dye or change the colour of the composition. The methods herein further provide an efficient means to neutralize the colours in a product. Neutralization may be particularly useful in pharmaceutical products, such as when white or dull-colour product is desired.

The colourant is added to the cannabis placebo compositions disclosed herein to provide a colour match to the active cannabis product. By “match”, it is meant that the colour is indistinguishable as between the cannabis placebo composition and the active cannabis product. In an embodiment, the colour as between the cannabis placebo composition and the active cannabis product are identical. In an embodiment, the colour as between the cannabis placebo composition and the active cannabis product are indistinguishable as measured subjectively, as described herein. In an embodiment, the colour as between the cannabis placebo composition and the active cannabis product are indistinguishable as measured objectively, as described herein.

To determine whether the cannabis placebo composition and the active cannabis product are objectively a match, a colorimeter may be used to measure the absorbance or transmittance. In another embodiment, a UV-Vis spectrophotometer may be used as described herein to obtain the absolute CIE XYZ values. Generally, if the deviation between the colour reading for the cannabis placebo composition and active cannabis product is less than 10%, 9%, 8%, 7%, 6%, 5%, 4%, 3%, 2%, 1%, 0.5%, 0.1%, or less, the cannabis placebo composition and the active cannabis product are a match in colour.

The cannabis placebo compositions as disclosed herein comprise one or more terpenes, esters, flavonoids, or any combination thereof to provide an odor and flavour match between the cannabis placebo composition and the active cannabis product.

Terpenes are a large and diverse class of organic compounds, produced by a variety of plants, particularly conifers, and by some insects such as termites or swallowtail butterflies, which emit terpenes from their osmeteria. They often have a strong odor and may protect the plants that produce them by deterring herbivores and by attracting predators and parasites of herbivores.

When terpenes are modified chemically, such as by oxidation or rearrangement of the carbon skeleton, the resulting compounds are generally referred to as terpenoids. The difference between terpenes and terpenoids is that terpenes are hydrocarbons, whereas terpenoids contain additional functional groups. As used herein, the term “terpene” encompasses terpenoids. Terpenoids are also known as isoprenoids. Any terpene can be converted to a terpenoid, synthetic terpenoid or semisynthetic terpenoid by an array of known chemical reactions. These conversions have been taught exhaustively in the art.

Terpenes and terpenoids are the primary constituents of the essential oils of many types of plants and flowers. Essential oils are used widely as fragrances in perfumery, and in medicine and alternative medicines such as aromatherapy. Synthetic variations and derivatives of natural terpenes and terpenoids also greatly expand the variety of aromas used in perfumery and flavors used in food additives. Terpenes are a major constituent of Cannabis sativa plants, which contain at least 120 identified compounds.

As used herein, the term “terpene” refers to a compound built on an isoprenoid structure or produced by combining isoprene units, 5 carbon structures. Within the context of this disclosure, the term “terpene” does not necessarily require 5 carbons or multiples of 5 carbons. It is understood that a reaction with isoprene units does not always result in a terpene comprising all the carbon atoms.

Within the context of this disclosure, the term “terpene” includes cannabis-derived terpenes and non-cannabis-derived terpenes. In a particular embodiment, the cannabis placebo composition herein comprises only cannabis-derived terpenes, without any terpenes from another source.

Within the context of this disclosure, the term “terpene” includes Hemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, or their derivatives. As well as isomeric, enantiomeric, or optically active derivatives.

Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids.

Within the context of this disclosure, the term terpene includes the α- (alpha), β- (beta), γ- (gamma), oxo-, isomers, or any combinations thereof.

Examples of terpenes within the context of this disclosure include, without limitation: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Alpha-Caryophyllene, Beta-Caryophyllene, Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/lcosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Herniarin, Hexanaldehyde, Hexanoic Acid, Hexyl acetate Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Pinene, Alpha-Pinene, Beta-Pinene, Propanethiol, Pristimerin, Pulegone, PhytolQuercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin.

In an embodiment, the cannabis placebo composition of the present disclosure comprises one or more terpenes selected from Beta-Myrcene, Linalool, Alpha-Pinene, Beta-Pinene, Beta-Caryophyllene, Caryophyllene oxide, Alpha-Humulene, Nerolidol, D-Limonene, L-Limonene, Para-Cymene, Eugenol, Farnesol, Geraniol, Phytol, Menthol, Terpineol, Alpha-Terpineol, Benzaldehyde, Hexyl acetate, Methyl Salicylate, Eucalyptol, Ocimene, Terpinolene, Alpha-Terpinene, Isopulegol, Guaicol, Alpha-Bisabolol, or any combination thereof.

As used herein, the term “ester” refers to any compound derived from an acid (organic or inorganic) in which at least one hydroxyl (—OH) group is replaced by an alkoxy (—O-alkyl) group. In an embodiment, the ester is Benzyl acetate, Benzyl formate, Ethyl acetate, Methyl acetylsalicylate, Methyl benzoate, or any combination thereof.

As used herein, the term “flavonoid” refers to any compound of a large class of plant pigments having a structure based on or similar to that of flavone. Flavonoids (from the Latin word flavus meaning yellow, their color in nature) are a class of plant and fungus secondary metabolites. Chemically, flavonoids have the general structure of a 15-carbon skeleton, which consists of two phenyl rings and a heterocyclic ring. This carbon structure can be abbreviated C6-C3-C6.

Within the context of this disclosure, the term “flavonoids” includes bioflavonoids, isoflavonoids (derived from 3-phenylchromen-4-one (3-phenyl-1,4-benzopyrone) structure) and neoflavonoids (derived from 4-phenylcoumarine (4-phenyl-1,2-benzopyrone) structure).

The three flavonoid classes above are all ketone-containing compounds, and as such, are anthoxanthins (flavones and flavonols). This class was the first to be termed bioflavonoids. The terms flavonoid and bioflavonoid have also been more loosely used to describe non-ketone polyhydroxy polyphenol compounds which are more specifically termed flavanoids. The three cycle or heterocycles in the flavonoid backbone are generally called ring A, B and C. Ring A usually shows a phloroglucinol substitution pattern.

Within the context of this disclosure, the term “flavonoid” includes anthocyanidins, anthoxanthins, flavanones, flavanonols and flavens.

Sources of flavonoids include, without limitation, cannabis, parsley, blueberries, black tea, citrus, wine, cocoa and peanut.

The cannabis placebo compositions may comprise one or more terpenes, esters, flavonoids, or any combination thereof to provide an odor and flavour match to the active cannabis product. By “match”, it is meant that the odor and flavour is indistinguishable as between the cannabis placebo composition and the active cannabis product. In an embodiment, the odor and flavour as between the cannabis placebo composition and the active cannabis product are identical. In an embodiment, the odor and flavour as between the cannabis placebo composition and the active cannabis product are indistinguishable as measured subjectively, as described herein. To determine whether the cannabis placebo composition and the active cannabis product are a match, a panel of subjects could be used to assess whether any differences in odor or flavour can be detected between the cannabis placebo composition and the active cannabis product. In an embodiment, this subjective testing is performed separately form the clinical trial established to study the effects of active agents.

In an embodiment, the cannabis placebo compositions comprise one or more terpenes to match a terpene profile of the active cannabis product. As used herein, “terpene profile” refers to the number, identity and quantity of terpenes in an active cannabis product. In an embodiment, the terpene profile includes a single terpene. In another embodiment, the terpene profile comprises two or more terpenes. As used herein, “to match a terpene profile” means in one embodiment to provide in the cannabis placebo composition the same terpenes at substantially the same quantity as in the active cannabis product. In another embodiment, “to match a terpene profile” means that different, but complementary terpenes, may be used. By “complementary” it is meant that individually or in sum the terpenes provide substantially the same smell and taste as provided by the terpenes of the active cannabis product.

In an embodiment, one exemplary group of terpenes that may be used in the cannabis placebo composition, together with one aroma associated therewith, includes: Aplha-Pinene, Pine; Beta-Pinene, Pine; Linalool, Lavender; Beta-Caryophyllene, Black pepper; Myrcene, Musk; Limonene, Citrus; Terpineol, Lilac; Nerolidol, Wood bark; Eucalyptol, Mint; Borneol, Camphor; Alpha-Bisabolol, Floral; Delta-3-Carene, Pine; and Camphene, Herbal.

In an embodiment, another exemplary group of terpenes that may be used in the cannabis placebo composition includes: Beta-Caryophyllene, Borneol, 1,8-Cineole, Camphene, Humulene, Limonene, Linalool, Myrcene, Nerolidol, Pulegone, and Terpinolene.

In an embodiment, another exemplary group of terpenes that may be used in the cannabis placebo composition includes: Alpha-Pinene, Beta-Pinene, Myrcene, Alpha-Phellandrene, Delta-3-Carene, Alpha-Terpinene, Beta-Phellandrene, Limonene, cis-Ocimene, Terpinolene, Beta-Caryophyllene, Alpha-Guaiene, Humulene, 8-Guaiene, Elemene, Guaiol, Gamma-Eudesmol, Beta-Eudesmol, Agarospirol, Bulnesol, and Alpha-Bisabolol.

In an embodiment, another exemplary group of terpenes includes: Alpha-Pinene, Linalool, Beta-Caryophyllene, Myrcene, and Limonene.

In an embodiment, another exemplary group of terpenes includes: Beta-Pinene, Nerolidol, Isopulegol and Gamma-Terpinne.

In another embodiment of terpenes to be used in the cannabis placebo composition herein, one or more individual terpenes described above may be assembled in additional suitable groups. In a particular embodiment, each member of the additional groups is independently selected from any of the group embodiments above.

In an embodiment, flavonoids for inclusion in the cannabis placebo composition of the present disclosure include, without limitation:
1. 6-OH-Luteolin 4′-methyl ether-7-(2″-a-rhamnoside-6′″-acetyl-b-glucoside);
2. 6-OH-Luteolin 7-(6″-(E)-caffeoyl)-b-glucoside;
3. Isoscutellarein 7-(2″-(6′″-acetyl)-b-allosyl)-b-glucoside;
4. Isoscutellarein 4′-methyl ether-7-(2″-(6′″-acetyl)-b-allosyl)-b-glucoside;
5. Apigenin 4′-(2″-(2′″-feruloyl-glucuronyl)-glucuronide);
6. Apigenin 7-glucuronide-4′-(2″-(2′″-feruloyl-glucuronyl)-glucuronide);
7. Apigenin 7-glucuronyl-4′-(2″-(2′″-E-p-coumaroyl-glucuronyl)-glucuronide);
8. Luteolin 3′-b-glucoside-4′-(2″-a-rhamnosyl-b-glucoside);
9. Luteolin 3′,4′-di-b-glucoside;
10. 5,7,4′-tri-OH-3′-OMe-Flavone 8-C-(2″-O-b-glucosyl-b-xyloside);
11. 5,7-di-OH-3′-OMe-4′-Acetoxyflavone 8-C-(2″-O-b-glucosyl-b-xyloside);
12. Iso-orientin 3′-methyl ether;
13. 8-C-p-OH-Benzoyl-isovitexin 4′-glucoside;
14. Apigenin 8-C-(2″-(4′″-acetyl-rhamnosyl)-glucoside);
15. Spinosin;
16. 6′″-Feruloyl-spinosin;
17. Isoscoparin 7-glucoside;
18. Carlinoside;
19. Kaempferol 3-(6″-a-arabinosyl-glucoside);
20. Kaempferol 3-(6″-a-arabinosyl-glucoside)-7-glucoside;
21. Kaempferol 3-(2″-rhamnosyl-6″-malonyl-glucoside);
22. Kaempferol 3-glucoside-7-(2″-(6′″-p-coumaroyl-glucosyl)-glucoside);
23. 8-OMe-Kaempferol 3-(6″-malonyl-glucoside);
24. Quercetin;
25. Quercetin 4′-glucoside;
26. Quercetin 3′-xyloside;
27. Myricetin 3-(2″-acetyl-rhamnoside);
28. Quercetin 3,4′-diglucoside;
29. Isorhamnetin 3-rutinoside;
30. Quercetin 3,7,4′-triglucoside;
31. Isorhamnetin 3,7-diglucoside;
32. Myricetin 3-(2″-rhamnosyl-glucoside);
33. Myricetin 3′-(6″-p-coumaroyl-glucoside);
34. Myricetin 7-(6″-galloyl-glucoside);
35. Laricitrin 3-a-arabinofuranoside;
36. Laricitrin 3-glucoside;
37. Syringetin 3-(5″-glucosyl-a-arabinofuranoside);
38. Syringetin 3-(6″-acetyl-glucoside);
39. Syringetin 3-robinobioside;
40. Syringetin 6-C-glucoside;
41. 6,3′-di-OH-4,4′-di-OMe-5-Me-Aurone;
42. 4,6,3′,4′-tetra-OMe-Aurone (Z-form);
43. 4,6,3′,4′-tetra-OMe-Aurone (E-form);
44. 6,3′,4′-tri-OH-4-OMe-5-Me-Aurone;
45. Maesopsin;
46. Maesopsin 6-O-glucoside (two diastereoisomers);
47. Licoagroaurone;
48. 3′-formyl-4′,6′-di-OH-2′-OMe-5-Me-Chalcone;
49. Chalcononaringenin 2′,4′-diglucoside;
50. 2′,4′-diOH-4′-OMe-6′-glucoside Dihydrochalcone;
51. 2′-OH-3′,4′,6′-tri-OMe-Dihydrochalcone;
52. Pelargonidin 3-glucoside-5-(6′″-acetyl-glucoside);
53. Pelargonidin 3-(6″-feruloyl-glucoside)-5-(6′″-malonyl-glucoside);
54. Cyanidin 3-(6″-malonyl-glucoside);
55. Cyanidin 3-rutinoside;
56. Cyanidin 3-(2″,3″-digalloyl-glucoside);
57. Cyanidin 3,4′-diglucoside;
58. Delphinidin 3-(6″-acetyl-galactoside);
59. Delphinidin 3′-(2″-galloyl-6″-acetyl-galactoside);
60. Peonidin 3-rutinoside;
61. Petunidin 3,7-diglucoside;
62. Petunidin 3-(6″-E-p-coumaroyl-glucoside)-5-(6′″-malonyl-glucoside);
63. Malvidin 3-(6″-E-p-coumaroyl-glucoside)-5-glucoside;
64. Malvidin 3-(6″-Z-p-coumaroyl-glucoside)-5-glucoside;
65. Malvidin 3-rutinoside-5-glucoside;
66. Malvidin 3-(6″-(4″″-malonyl-rhamnosyl)-glucoside)-5-(6′″-malonyl-glucoside);
67. Apigeninidin 5-glucoside;
68. Luteolinidin 5-glucoside;
69. Carboxypyrano Pelargonidin 3-glucoside;
70. Carboxypyrano Cyanidin 3-glucoside;
71. Carboxypyrano Cyanidin 3-(6″-malonyl-glucoside;)
72. Carboxypyrano Malvidin 3-glucoside;
73. Judaicin 7-(6″-acetyl-glucoside);
74. Tectorigenin 4′-(6″-glucosyl-glucoside);
75. 7-OH-6′-OMe-3′,4′-methylenedioxyisoflavone 7-glucoside;
76. Irisjaponin A;
77. Irisjaponin B;
78. Junipegenin B;
79. Matteucinol 7-(6″-apiofuranosyl-b-glucoside);
80. Hesperitin 7-(2″-galactosyl-6″-rhamnosyl-glucoside);
81. Persicogenin 5,3′-di-OH-7,4′-di-OMe-flavanone;
82. Naringenin 7-glucoside;
83. Naringenin 7-(6″-galloyl-glucoside);
84. Taxifolin 4′-glucoside;
85. Aromadendrin 7-glucoside;
86. Ampelopsin 7-glucoside;
87. 2″-Accallunin;
88. 2R,3R-trans-aromadendrin 7-(6″-(4′″-OH-2′″-methylenebutanoyl)-glucoside);
89. (2R,3S)-( [symbol] )-3′,5-di-OH-4′,7-di-OMe-Dihydroflavonol;
90. 3-Desoxycallunin;
91. Catechin 3-(6″-cinnamoyl-glucoside);
92. Catechin 3-(2″-cinnamoyl-glucoside);
93. Catechin 3-(2″,6″-dicinnamoyl-glucoside);
94. Anadanthoside;
95. Cajanin;
96. Indicanine C;
97. 6-(1,1-di-Me-allyl)-7,4′-di-OH-Flavan;
98. 3-(4′-hydroxyphenyl)-5-methoxy-6-(3,3-dimethylallyl)-2″,2″-dimethylchromene-(5″,6″:8,7)-3-(propyl-2-one)-4H-1-benzo-2,3-Dihydropyran-2,4-dione;
99. Maackianin 3-(6″-malonyl-glucoside);
100. 3,4:8,9-Dimethylenedioxy-pterocarpan;
101. Usararotenoid C;
102. 12a-Epimillettosin;
103. ( [symbol] )-Usararotenoid-B;
104. [Catechin 3-glucoside-(4a->8)-catechin 3-(2″-cinnamoyl-glucoside)];
105. [Catechin 3-glucoside-(4a->8)-epicatechin 3-(6″-cinnamoyl-glucoside)];
106. Amentoflavone;
107. Aulacomnium-biaureusidin;
108. Cupressuflavone 7,7″-dimethyl ether;
109. 4,4′,6-tri-O-methyl-2-deoxymaesopsin-(2->7)-2,4,4′,6-tetra-O-Methylmaesopsin;
110. Catechin-(4a->8)-pelargonidin 3-glucoside;
111. 2′,2″,2′″-tri-OH-4′,4′″-di-OMe-4-O-5′″-bichalcone (Rhuschalcone 1);
112. Puerarin (Daidzein 8-C-glucoside);
113. Calycosin;
114. Isoneorautenol; and
115. Erybraedin A.

In an embodiment, flavonoids for inclusion in the cannabis placebo composition as disclosed herein include any one or more of flavonoids 1 to 115 above, each selected independently from the above list.

In an embodiment, another exemplary group of flavonoids that may be used in the cannabis placebo composition includes: Apigenin, Beta-Sitosterol, Orientin, Quercitrin, Apigenin-7-O-glucoside, Luteolin, Apigenin, Kaempferol, Cannflavin A, Cannflavin B, Myricetin, and Luteolin-7-O-glucoside.

In an embodiment, another exemplary group of flavonoids that may be used in the cannabis placebo composition includes: Quercitrin, Apigenin-7-O-glucoside, Luteolin, Apigenin, Kaempferol, Cannflavin A, Cannflavin B, Myricetin, and Luteolin-7-O-glucoside.

In another embodiment of flavonoids to be used in the cannabis placebo composition herein, one or more individual flavonoids described above may be assembled in additional suitable groups. In a particular embodiment, each member of the additional groups is independently selected from any of the group embodiments above.

In an embodiment, the cannabis placebo composition as disclosed herein consists of only naturally derived components. By “naturally derived” it is meant that the compound is obtained from a natural source, rather than by synthetic or semisynthetic means. In an embodiment, the components are extracted and/or purified from the natural source. In an embodiment, the natural source is a plant source.

In a particular and exemplary embodiment, the cannabis placebo composition of the present disclosure comprises MCT oil, Beta-Carotene extract, Alfalfa extract and two or more cannabis-derived terpenes. In an embodiment thereof, the cannabis-derived terpenes in the cannabis placebo compositions match a terpene profile of the active cannabis product, as described elsewhere herein.

In a particular and exemplary embodiment, the cannabis placebo composition of the present disclosure consists of MCT oil, Beta-Carotene extract, Alfalfa extract and two or more cannabis-derived terpenes. In an embodiment thereof, the cannabis-derived terpenes in the cannabis placebo compositions match a terpene profile of the active cannabis product, as described elsewhere herein.

The cannabis placebo composition of the present disclosure may be prepared by any suitable means, including combining the components described herein (e.g. base components, colourant, and terpenes, esters and flavonoids) in any order. In a particular, embodiment, the base components and the terpenes, esters and/or flavonoids are combined, and then the colourants are added in accordance with the methods described herein to provide a colour-matched product.

As will be appreciated from the above, in the context of the present disclosure a “cannabis placebo composition” is either a placebo that: (i) has no intended therapeutic, medicinal and recreational effects, or (ii) has at least one observable effect at a controlled level to mimic an observable effect of an active cannabis product, whereby the mimicked observable effect is not the effect of the active agent(s) under study. The observable effect found in the cannabis placebo composition is preferably distinguishable from any intended therapeutic, medicinal or recreational effect under study in the active cannabis product. In an embodiment, the cannabis placebo composition as disclosed herein is capable of mimicking all observable effects of the active cannabis product, except the effect that is under study.

Cannabis placebo compositions of the present disclosure that have no therapeutic, medicinal or recreational effects are, in effect, an “inert” composition in that it lacks any compounds that should cause a biological effect in a subject. Notably however, in view of the placebo effect, this does not necessarily mean that the cannabis placebo composition will not cause an effect in particular subjects. In fact, this is the purpose of a placebo—to observe the importance and effects of perception and the conversion of psychological responses into biological responses.

In an embodiment of the so-called inert compositions of the present disclosure, the cannabis placebo compositions are devoid of cannabinoids. In an embodiment, the inert compositions are likewise or alternatively devoid of terpenes that have observable biological effects aside from providing flavour and/or odor to the cannabis placebo compositions disclosed herein. Thus, in an embodiment, the cannabis placebo compositions do not comprise any compounds of a cannabis plant or extract thereof that would be considered a therapeutically or recreationally active compound.

The so-called inert cannabis placebo compositions of the present disclosure are particularly advantageous in studying the therapeutic, medicinal or recreational effects of active agents in a corresponding active cannabis product that does not have psychoactive effects. In this manner, the cannabis placebo composition is indistinguishable from the active cannabis product in colour, odor and flavour, and the subject does recognize the difference between the two products based on any psychoactive effect that is not under study. Thus, the cannabis placebo composition herein functions as a placebo to the active cannabis product, and the actual effect of the active agents can be studied having regard to any placebo effect.

A cannabis or a cannabis-derived composition (e.g. cannabis oil) may be subjected to purification, separation or other means of refinement such that one or more compounds (e.g. cannabinoids) present in the cannabis or cannabis-derived composition have been modified in the final active cannabis product. In addition or in the alternative, one or more compounds (e.g. active agents) may be added to the active cannabis product. For example, the presence, absence or amount of a compound may be changed in the active cannabis product as compared to the original cannabis or cannabis-derived composition. In an embodiment, an additional quantity of one or more cannabinoids may be added to the active cannabis product (e.g. primary cannabinoids).

As used herein, the term “cannabinoid” refers to a compound belonging to a class of secondary compounds commonly found in plants of genus cannabis. In many cases, a cannabinoid can be identified because its chemical name will include the text string “*cannabi* in the name. However, there are a number of cannabinoids that do not use this nomenclature, such as for example those described herein.

A cannabinoid is one of a class of diverse chemical compounds that acts on cannabinoid receptors such as CB1 and CB2 in cells that alter neurotransmitter release in the brain. Ligands for these receptor proteins include the endocannabinoids (produced naturally in the body by animals), the phytocannabinoids (found in cannabis and some other plants), and synthetic cannabinoids (manufactured artificially). The most notable cannabinoid of the phytocannabinoids is tetrahydrocannabinol (THC), the primary psychoactive compound in cannabis. Cannabidiol (CBD) is another cannabinoid that is a major constituent of the plant. There are at least 113 different cannabinoids isolated from cannabis, exhibiting varied effects.

Synthetic cannabinoids and semisynthetic cannabinoids encompass a variety of distinct chemical classes: the classical cannabinoids structurally related to THC, the non-cannabinoids (cannabimimetics) including the aminoalkylindoles, 1,5-diarylpyrazoles, quinolines, and arylsulfonamides, as well as eicosanoids related to endocannabinoids.

Within the context of this application, where reference is made to a particular cannabinoid, each of the acid and/or decarboxylated forms are contemplated as both single molecules and mixtures.

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Tetrahydrocannabinolic acid C4 (THCA-C4), Tetrahydrocannbinol C4 (THC C4), Tetrahydrocannabivarinic acid (THCVA), Tetrahydrocannabivarin (THCV), Δ8-Tetrahydrocannabivarin (Δ8-THCV), Δ9-Tetrahydrocannabivarin (Δ9-THCV), Tetrahydrocannabiorcolic acid (THCA-C1), Tetrahydrocannabiorcol (THC-C1), Delta 7 cis iso tetrahydrocannabivarin, Δ8-tetrahydrocannabinolic acid (Δ8-THCA), Δ8-tetrahydrocannabinol (Δ8-THC), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11-nor-9-carboxy-Δ9-tetrahydrocannabinol, Ethoxy-cannabitriolvarin (CBTVE), 10-Ethoxy-9-hydroxy-Δ6a-tetrahydrocannabinol, Cannabitriolvarin (CBTV), 8,9-Dihydroxy-Δ6a(10a)-tetrahydrocannabinol (8,9-Di-OH-CBT-05), Dehydrocannabifuran (DCBF), Cannbifuran (CBF), Cannabichromanon (CBCN), Cannabicitran (CBT), 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E,4E-dienoic acid isobutylamide.

THC refers to a psychotropic cannabinoid and is the principal psychoactive constituent of cannabis. Its chemical name is (−)-trans-Δ9-tetrahydrocannabinol and the term “THC” is used to refer to isomers as well. Like most pharmacologically-active secondary metabolites of plants, THC in cannabis is assumed to be involved in self-defense, perhaps against herbivores. THC also possesses high UV-B (280-315 nm) absorption properties, which, it has been speculated, could protect the plant from harmful UV radiation exposure.

CBD is one of the active cannabinoids identified in cannabis. It is a major phytocannabinoid, by some accounts making up to 40% of the plant's extract. CBD does not appear to have any intoxicating effects such as those caused by THC in marijuana, but may have effects on anxiety, depression and have an anti-psychotic effect, and have effects on other comorbidities. In some instances, the comorbidities are related to disorders such as pain and post-traumatic stress disorder, commonly referred to as “PTSD”.

CBN is thought to be a non-psychoactive cannabinoid found only in trace amounts in cannabis and can be produced via oxidative degradation of THCA and THC. Pharmacologically relevant quantities are formed as a metabolite of THC. CBN acts as a partial agonist at the CB1 receptors, but has a higher affinity to CB2 receptors, however; with lower affinities in comparison to THC. Degraded or oxidized cannabis products, such as low-quality baled cannabis and traditionally produced hashish, are high in CBN, but modern production processes have been alleged to minimize the formation of CBN. CBN has been shown to have analgesic properties. Unlike other cannabinoids, CBN does not stem from cannabigerol (CBG).

CBG is thought to be a non-intoxicating cannabinoid found in the Cannabis genus of plants. CBG is the non-acidic form of cannabigerolic acid (CBGA), the parent molecule (“mother cannabinoid”) from which many other cannabinoids are obtained. CBG has been found to act as a high affinity α2-adrenergic receptor agonist, moderate affinity 5-HT1A receptor antagonist, and low affinity CB1 receptor antagonist. It also binds to the CB2 receptor as an antagonist. CBG does not trigger THC-like activity in mice, rats, gerbils and non-human primates, consistent with it being non-intoxicating. Moreover, CBG was without effect up to 80 mg/kg in the mouse tetrad test of cannabimimetic activity (locomotor suppression, catalepsy, hypothermia and analgesia).

CBGA is the alleged primordial phytocannabinoid. It is the alleged compound in cannabis from which all the plant's other naturally occurring cannabinoids are formed; without CBGA, the cannabis plant cannot produce its most useful compounds. It remains one of the most under-studied cannabinoids, with most of current research focusing on the purported healing properties of THC and CBD.

As used herein, the term “THC” refers to tetrahydrocannabinol and has the following structural formula:

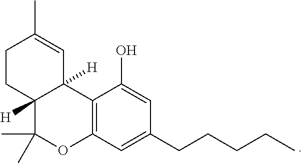 [SMILES: CCCCCc1cc(O)c2c(c1)OC(C)(C)[C@@H]1CCC(C)=C[C@@H]21]

As used herein, “THC” is used interchangeably with “Δ9-THC”.

As used herein, the term “THCA” refers to tetrahydrocannabinolic acid and has the following structural formula:

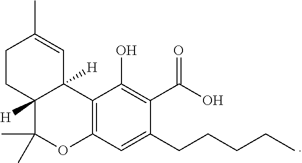 [SMILES: CCCCCc1cc2c(c(O)c1C(=O)O)[C@@H]1C=C(C)CC[C@H]1C(C)(C)O2]

Decarboxylating THCA with heat, light, etc., forms THC, Δ8-THC, and other potential cannabinoids.

As used herein, the term “THCV” refers to tetrahydrocannabivarin and has the following structural formula:

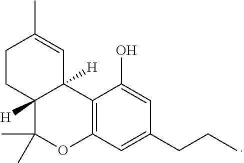 [SMILES: CCCc1cc(O)c2c(c1)OC(C)(C)[C@@H]1CCC(C)=C[C@@H]21]

As used herein, the term “THCVA” refers to tetrahydrocannabivarinic acid and has the following structural formula:

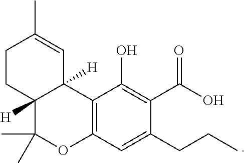 [SMILES: CCCc1cc2c(c(O)c1C(=O)O)[C@@H]1C=C(C)CC[C@H]1C(C)(C)O2]

Decarboxylating THCVA with heat, light, etc., forms THCV, Δ8-THCV, Δ9-THCV, and other possible cannabinoid derivatives.

As used herein, the term “Δ8-THC” refers to delta-8-tetrahydrocannabinol and has the following structural formula:

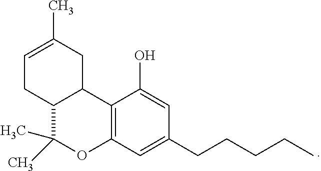

As used herein, the term “Δ8-THCV” refers to delta-8-tetrahydrocannabivarin and has the following structural formula:

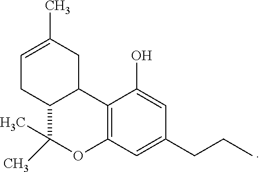

As used herein, the term “Δ9-THCV” refers to delta-9-tetrahydrocannabivarin and has the following structural formula:

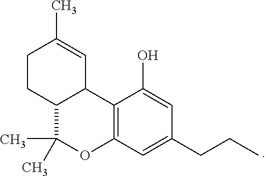

As used herein, the term “CBD” refers to cannabidiol and has the following structural formula:

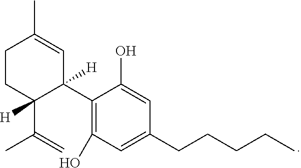 [SMILES: C=C(C)[C@@H]1CCC(C)=C[C@H]1c1c(O)cc(CCCCC)cc1O]

As used herein, the term “CBDA” refers to cannabidiolic acid and has the following structural formula:

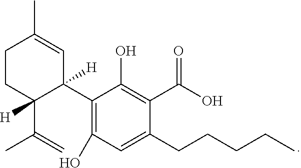 [SMILES: C=C(C)[C@@H]1CCC(C)=C[C@H]1c1c(O)cc(CCCCC)c(C(=O)O)c1O]

Decarboxylating CBDA with heat, light, etc., forms CBD and other possible cannabinoid derivatives.

As used herein, the term “CBDV” refers to cannabidivarin and has the following structural formula:

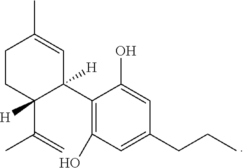 [SMILES: C=C(C)[C@@H]1CCC(C)=C[C@H]1c1c(O)cc(CCC)cc1O]

As used herein, the term “CBDVA” refers to cannabidivarinic acid and has the following structural formula:

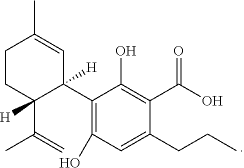 [SMILES: C=C(C)[C@@H]1CCC(C)=C[C@H]1c1c(O)cc(CCC)c(C(=O)O)c1O]

Decarboxylating CBDVA with heat, light, etc., forms CBDV and other possible cannabinoid derivatives.

As used herein, the term “CBC” refers to cannabichromene and has the following structural formula:

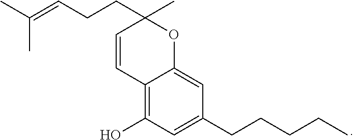 [SMILES: CCCCCc1cc(O)c2c(c1)OC(C)(CCC=C(C)C)C=C2]

As used herein, the term “CBCA” refers to cannabichromenic acid and has the following structural formula:

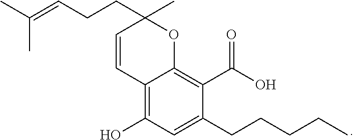 [SMILES: CCCCCc1cc(O)c2c(c1C(=O)O)OC(C)(CCC=C(C)C)C=C2]

Decarboxylating CBCA with heat, light, etc., forms CBC and other possible cannabinoid derivatives.

As used herein, the term “CBCV” refers to cannabichromevarin and has the following structural formula:

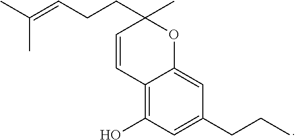 [SMILES: CCCc1cc(O)c2c(c1)OC(C)(CCC=C(C)C)C=C2]

As used herein, the term “CBCVA” refers to cannabichromevarinic acid and has the following structural formula:

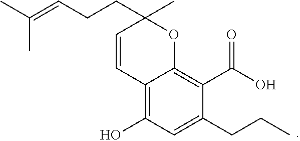 [SMILES: CCCc1cc(O)c2c(c1C(=O)O)OC(C)(CCC=C(C)C)C=C2]

Decarboxylating CBCVA with heat, light, etc., forms CBCV and other possible cannabinoid derivatives.

As used herein, the term “CBG” refers to cannabigerol and has the following structural formula:

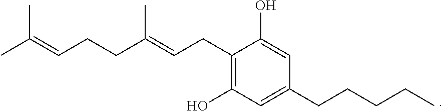 [SMILES: CCCCCc1cc(O)c(C/C=C(\C)CCC=C(C)C)c(O)c1]

As used herein, the term “CBGA” refers to cannabigerolic acid and has the following structural formula:

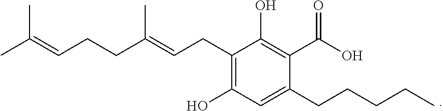 [SMILES: CCCCCc1cc(O)c(C/C=C(\C)CCC=C(C)C)c(O)c1C(=O)O]

Decarboxylating CBGA with heat, light, etc., forms CBG and other possible cannabinoid derivatives.

As used herein, the term “CBGV” refers to cannabigerovarin and has the following structural formula:

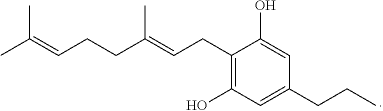 [SMILES: CCCc1cc(O)c(C/C=C(\C)CCC=C(C)C)c(O)c1]

As used herein, the term “CBGVA” refers to cannabigerovarinic acid and has the following structural formula:

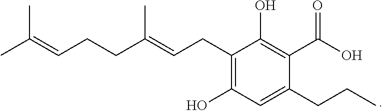 [SMILES: CCCc1cc(O)c(C/C=C(\C)CCC=C(C)C)c(O)c1C(=O)O]

Decarboxylating CBGVA with heat, light, etc., forms CBGV and other possible cannabinoid derivatives.

As used herein, the term “CBN” refers to cannabinol and has the following structural formula:

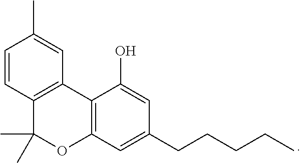 [SMILES: CCCCCc1cc(O)c2c(c1)OC(C)(C)c1ccc(C)cc1-2]

As used herein, the term “CBNA” refers to cannabinolic acid and has the following structural formula:

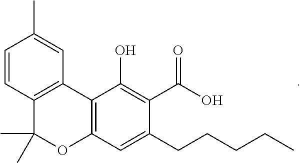 [SMILES: CCCCCc1cc2c(c(O)c1C(=O)O)-c1cc(C)ccc1C(C)(C)O2]

Decarboxylating CBNA with heat, light, etc., forms CBN and other possible cannabinoid derivatives.

As used herein, the term “CBNV” or “CBV” refers to cannabivarin and has the following structural formula:

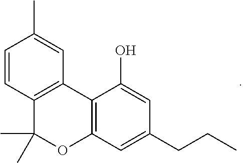 [SMILES: CCCc1cc(O)c2c(c1)OC(C)(C)c1ccc(C)cc1-2]

As used herein, the term “CBNVA” refers to cannabivarinic acid and has the following structural formula:

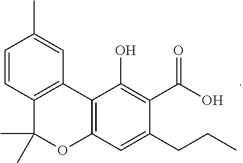 [SMILES: CCCc1cc2c(c(O)c1C(=O)O)-c1cc(C)ccc1C(C)(C)O2]

Decarboxylating CBNVA with heat, light, etc., forms CBNV and other possible cannabinoid derivatives.

As used herein, the term “CBND” refers to cannabinodiol and has the following structural formula:

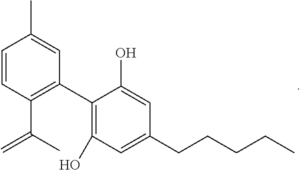 [SMILES: C=C(C)c1ccc(C)cc1-c1c(O)cc(CCCCC)cc1O]

As used herein, the term “CBNDA” refers to cannabinodiolic acid and has the following structural formula:

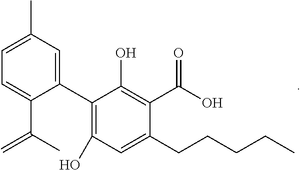 [SMILES: C=C(C)c1ccc(C)cc1-c1c(O)cc(CCCCC)c(C(=O)O)c1O]

Decarboxylating CBNDA with heat, light, etc., forms CBND and other possible cannabinoid derivatives.

As used herein, the term “CBNDV” refers to cannabivarinodiol and has the following structural formula:

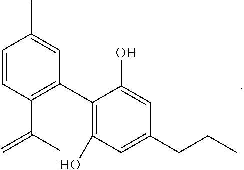 [SMILES: C=C(C)c1ccc(C)cc1-c1c(O)cc(CCC)cc1O]

As used herein, the term “CBNDVA” refers to cannabivarinodiolic acid and has the following structural formula:

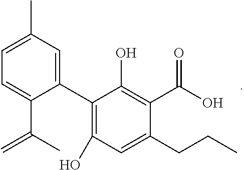 [SMILES: C=C(C)c1ccc(C)cc1-c1c(O)cc(CCC)c(C(=O)O)c1O]

Decarboxylating CBNDVA with heat, light, etc., forms CBNDV and other possible cannabinoid derivatives.

As used herein, the term “CBL” refers to cannabicyclol and has the following structural formula:

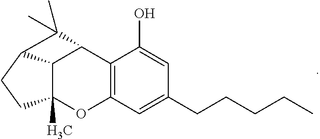

As used herein, the term “CBLA” refers to cannabicyclolic acid and has the following structural formula:

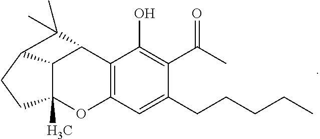

Decarboxylating CBLA with heat, light, etc., forms CBL and other possible cannabinoid derivatives.

As used herein, the term “CBLV” refers to cannabicyclovarin and has the following structural formula:

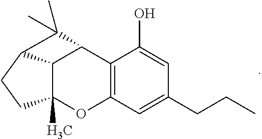

As used herein, the term “CBLVA” refers to cannabielvarinsoinic acid and has the following structural formula:

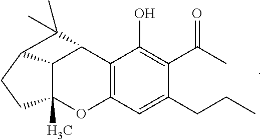

Decarboxylating CBLVA with heat, light, etc., forms CBLV and other possible cannabinoid derivatives.

As used herein, the term “CBE” refers to cannabielsoin and has the following structural formula:

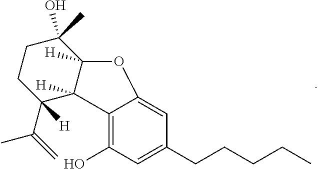

As used herein, the term “CBEA” refers to cannabielsoic acid and has the following structural formula:

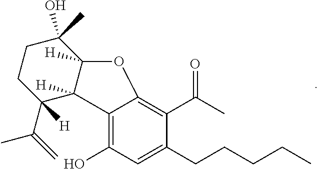

Decarboxylating CBEA with heat, light, etc., forms CBE and other possible cannabinoid derivatives.

As used herein, the term “CBEV” refers to cannabivarinselsoin and has the following structural formula:

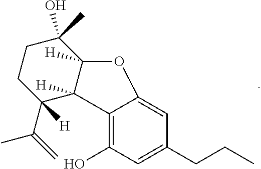

As used herein, the term “CBEVA” refers to cannabivarinselsoinic acid and has the following structural formula:

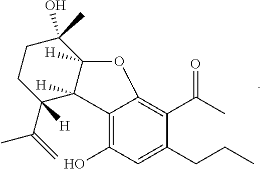

Decarboxylating CBEVA with heat, light, etc., forms CBEV and other possible cannabinoid derivatives.

In the context of the present disclosure, the primary cannabinoid under study in the active cannabis product may be any one or more of the cannabinoids as disclosed herein.

In a particular embodiment, the primary cannabinoid under study in the active cannabis product may be selected from Cannabigerol-type (CBG): cannabigerol ((E)-CBG _{C-5}), cannabigerol monomethyl ether ((E)-CBGM _{C-5} A), Cannabinerolsäure A ((Z)-CBGA _{C-5} A), Cannabigerovarin (((e)-CBGVC _{C-3}), Cannabigerolsäure A (e)-CBGA _{C-5} A), A Cannabigerolsäure monomethyl ether ((e)-CBGAM _{C-5} A), Cannabigerovarinsäure A ((e)-CBGVA-C ₃ A); Cannabichromene-type (CBC): cannabichromene (CBC-C 5), Cannabichromensäure A (CBCA _{C-5} A), Cannabichromevari n_{(CBCVC-3)}, Cannabichromevarinsäure A (CBCVA-C3 A); Cannabidiol-type (CBD):, cannabidiol (CBD-C ₅₎, cannabidiol monomethyl (CBDM-C ₅₎, cannabidiol-C4 (CBD-C ₄₎, Cannabidivarin (CBDV-C ₃₎, Cannabidiorcol (CBD-C ₁₎, cannabidiolic (CBDA C-5), Cannabidivarinsäure (CBDVA _{C-3}), Cannabinodiol-like (CBND): Cannabinodiol (CBND C-5), Cannabinodivarin (CBND C-3); Tetrahydrocannabinol-like (THC): Δ9-tetrahydrocannabinol (Δ9-THC-C ₅₎, Δ9-tetrahydrocannabinol-C4 (Δ9-THC-C ₄₎, Δ9-tetrahydrocannabivarin (Δ9-THCV-C ₃₎, Δ9-Tetrahydrocannabiorcol (Δ9-THCO _{C-1)}, Δ9-Tetrahydrocannabinolsäure (Δ9 _{THCA-C-5} A), Δ9-Tetrahydrocannabinolsäure B (ϕ9 _{THCA-C-5} B), Δ9-Tetrahydrocannabinolsäure-C4 (Δ9 _{THCA-C-4} A and/or B), Δ9-Tetrahydrocannabivarinsäure A (Δ9-THCVA-C ₃ A), Δ9-Tetrahydrocannabiorcolsäure (Δ9-THCOA-C ₁ A and/or B), (−)-Δ8-trans-(6aR, 10aR)-Δ8-tetrahydrocannabinol (Δ8-THC-C ₅₎, (−)- Δ8-trans-(6aR, 10aR)-Tetrahydrocannabinolsäure A (Δ8-THCA-C ₅ A); (−)-(6a S, 10a R)-Δ9-tetrahydrocannabinol ((−)-cis-Δ9-THC-C ₅); Cannabinol-type (CBN): Cannabinol CBN-C ₅, cannabinol C4 _{(CBN-C4)}, Cannabivarin (CBN-C ₃₎, cannabinol C2 (CBN-C ₂₎, Cannabiorcol (CBN-C ₁₎, Cannabinolsäure A (C ₅ CBNA-A), Cannabinolmethylether (CBNM _{C-5)} Cannabitriol-type (CBT): (−)-(9R, 10R)-trans-Cannabitriol ((−)-trans-CBT-C ₅₎, (+)-(9S, 10S)-Cannabitriol ((+)-trans-CBT _{C-5)}, (±)-(9R, 10S/9S, 10R)-Cannabitriol ((±)-cis-CBT-C ₅₎, (−)-(9R, 10R)-trans [10-0-ethyl-cannabitriol]((−)-trans-CBT-OEt-C ₅₎, (±)-(9R, 10R/9S, 10S)-Cannabitriol-C3 ((±)-trans-CBT-C ₃₎, 8,9-dihydroxy-Δ6a (10a) tetrahydrocannabinol (8,9-di-OH-CBT-C ₅₎, cannabidiolic A (CBDA C-59-OH-CBT-05 ester), (−)-(6aR, 9S, 10S, 10aR)-9,10-dihydroxy-hexahydrocannabinol, Cannabiripsol Cannabiripsol-C5, (−6a, 7,10a-trihydroxy-Δ9-tetrahydrocannabinol ((−)-Cannabitetrol), 10-oxo-Δ6a (10a) tetrahydrocannabinol (OTHC); Cannabielsoin-like (CBE): (5aS, 6S, 9R, 9aR)-C ₅-Cannabielsoin (CBEC-5), (5aS, 6S, 9R, 9aR)-C ₃-Cannabielsoin (CBE _{C-3)}, 5aS, 6S, 9R, 9aR)-Cannabielsoinsäure A (CBEA-C ₅A), (5aS, 6S, 9R, 9aR)-Cannabielsoinsäure B (CBEA-C ₅ B), (5aS, 6S, 9R, 9aR)-C3-Cannabielsoinsäure B (CBEA-C ₃ B), Cannabiglendol-C3 (OH-iso-HHCV _{C-3)}, Dehydrocannabifuran (DCBF _{C-5)}, Cannabifuran (CBF-C ₅₎; lsocannabinoide: (−)-Δ7-trans-(1R, 3R, 6R)-Isotetrahydrocannabinol, (±)-Δ7-1,2-cis- (1R, 3R, 6S/1S, 3S, 6R)-Isotetrahydrocannabivarin, (−)-47-trans-(1R, 3R, 6R)-Isotetrahydrocannabivarin; Cannabicyclol-like (CBL): (±)-(1aS, 3aR, 8bR, 8Cr-cannabicyclol (CBL-C ₅₎, (±)-(1aS, 3aR, 8bR, 8Cr-Cannabicyclolsäure A (CBLA-C 5A) (±)-(1aS, 3aR, 8bR, 8Cr-Cannabicyclovarin (CBLV _{C-3)}, Cannabicitran-type (CBT): Cannabicitran (CBT-C ₅₎; Cannabichromanon-like (CBCN): Cannabichromanon (CBCN C-5), Cannabichromanon-C3 (CBCN _{C-3)}, Cannabicoumaronon (CBCON C-5).

In addition to the above cannabinoids, the carboxylic acids which are biosynthetic precursors of each are contemplated as cannabinoids of the disclosure.

In a particular embodiment, the primary cannabinoid under study in the active cannabis product may be selected from THC, Δ8-THC, THCA, THCV, Δ8-THCA, Δ9-THCA, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, CBLVA, or any combination thereof.

In a particular embodiment, the primary cannabinoid under study in the active cannabis product may be selected from THC, CBD, CBG, CBN, CBC, THCV, CBGA, CGCA, CBCA, THCA and CBDA.

In a particular embodiment, the primary cannabinoid under study in the active cannabis product may be selected from THC, CBD, CBG, CBN, and CBC.

In an embodiment, the present disclosure is directed to a cannabis placebo composition to act as a placebo for an active cannabis product comprising any one or more of the primary cannabinoids described herein. In an embodiment, one or more of the primary cannabinoids have been modified, e.g. in their presence, absence or amount.

The so-called inert cannabis placebo compositions is useful as a placebo against any active cannabis product that does not result in an observable effect, other than those effects under study. In an embodiment, the active cannabis product does not include secondary cannabinoids as defined herein. In certain aspects of this embodiment, none of the primary cannabinoids in the active cannabis product are psychoactive cannabinoids. In other aspects of this embodiment, one or more of the primary cannabinoids are psychoactive cannabinoids, but it is the effect of these cannabinoids that is under study.

The so-called inert cannabis placebo compositions of the present disclosure may also be useful as a delivery vehicle for one or more active agents. In such embodiments, an active agent can be incorporated into the cannabis placebo composition to generate an active product having therapeutic, medicinal or recreational effect. Such embodiments may be particularly useful in delivering active agents to subjects who enjoy the smell and taste of cannabis. In such instances, the delivery vehicle disclosed herein may provide an effective means to increase a subject's desire or willingness to take a drug and, in turn, may increase compliance with treatment regimes.

In the event that an active cannabis product under study has more than one biologically observable effect, including one or more effects observable by a test subject that are not an effect under study, it is important to use a placebo of the present disclosure that mimics that effect which is not under study. By way of example, if an active cannabis product under study has two biologically observable effects on a test subject, and the active cannabis product is being studied for only one of those effects, the cannabis placebo composition of the present disclosure is designed to present the subject with the effect not being studied such that the placebo and active product are indistinguishable. When there are multiple effects of the active cannabis product under study, the design, making and using of the cannabis placebo composition of the present disclosure relies on providing the subject with ingredients which mimic all of the observable effects of the active cannabis product, except that of the effect under study.

In one particular embodiment, the ratio of THC to CBD may be modified in the active cannabis product and the cannabis placebo composition may be used to observe the therapeutic effect of the ratio change. In such embodiments, it may be appropriate to include in the cannabis placebo herein one or more psychoactive compounds to mimic the psychoactive effect of THC so the placebo and active cannabis product are indistinguishable. In an embodiment, a comparable amount of THC may be included in the cannabis placebo composition. In another embodiment, another psychoactive substance or substances may be included in the cannabis placebo composition, whereby the psychoactive substance(s) mimic the effect of THC in the active cannabis product.

As used herein, a “psychoactive substance” is a substance, compound, or drug that acts primarily on the central nervous system where it alters brain function, resulting in temporary changes in perception, mood, consciousness and/or behaviour. These substances may, for example, be used recreationally to purposefully alter one's consciousness (such as coffee, alcohol or cannabis), as entheogens for spiritual purposes (such as the mescaline-containing peyote cactus or psilocybin-containing mushrooms), as medication (such as the use of narcotics in controlling pain, stimulants to treat narcolepsy and attention disorders, as well as anti-depressants and anti-psychotics for treating neurological and psychiatric illnesses), and for research.

Some categories of psychoactive drugs have therapeutic value, and are prescribed by physicians and other healthcare practitioners. Examples include anesthetics, analgesics, anticonvulsant and antiparkinsonian drugs, as well as medications used to treat neuropsychiatric disorders, such as antidepressants, anxiolytics, antipsychotics, and stimulant medications. Some psychoactive substances may be used in the detoxification and rehabilitation programs for persons dependent on or addicted to other psychoactive drugs.

Psychoactive substances often bring about subjective (although these may be objectively observed) changes in consciousness and mood that the user may find rewarding and pleasant (e.g., euphoria or a sense of relaxation) or otherwise advantageous (e.g. increased alertness), and are thus reinforcing. Substances which are both rewarding and positively reinforcing have the potential to induce a state of addiction—compulsive drug use despite negative consequences. In addition, sustained use of some substances may produce physical or psychological dependence or both, associated with somatic or psychological-emotional withdrawal states respectively. Drug rehabilitation attempts to reduce addiction, through a combination of psychotherapy, support groups, and other psychoactive substances. Conversely, certain psychoactive drugs may be so unpleasant that the person will never use the substance again. This is especially true of certain deliriants (e.g. Jimson weed), powerful dissociatives (e.g. Salvia divinorum), and classic psychedelics (e.g. LSD, psilocybin), in the form of a “bad trip”.

Within the context of the present disclosure, psychoactive substances and the like can be either desired, undesired, or unimportant constituents of the active cannabis product herein described. It is possible a psychoactive substance or the like can have a beneficial impact on the utility of the compositions in treating diseases or disorders. It is also possible that psychoactive substances or the like can create unwanted side effects of treating diseases or disorders such as causing euphoria, anxiety, or intoxication during treatment of the disease or disorder.

In an embodiment, the psychoactive substance to be included in the cannabis placebo composition is a secondary cannabinoid. In an embodiment, the psychoactive substance to be included in the cannabis placebo composition is non-cannabinoid drug that has psychoactive properties that can mimic the psychoactive properties of cannabinoids.

In contrast to the so-called inert cannabis placebo compositions described above, in another embodiment the cannabis placebo compositions disclosed herein may comprise one or more secondary cannabinoids. As discussed above, a secondary cannabinoid is a cannabinoid that is present in both the cannabis placebo composition and the active cannabis product. As such, the effect of the secondary cannabinoid is not under study against the placebo. These types of placebo compositions are advantageous for studying the effect of active agents in combination with cannabinoids. The active agent may itself be a cannabinoid (“primary cannabinoid”) or may by a different active ingredient, such as a drug. The active agent under study is not included in the cannabis placebo composition.

In particularly advantageous embodiments, the secondary cannabinoids in the cannabis placebo composition are psychoactive cannabinoids and thereby render the placebo indistinguishable from an active cannabis product that contains psychoactive cannabinoids in combination with other active agents under study.

The secondary cannabinoids are present within the cannabis placebo composition at a controlled amount to provide an effect that is indistinguishable from the active cannabis product. In an embodiment, the secondary cannabinoids in the cannabis placebo composition match a secondary cannabinoid profile in the active cannabis product. As used herein, “secondary cannabinoid profile” refers to the number, identity and quantity of secondary cannabinoids in an active cannabis product. In an embodiment, the secondary cannabinoid profile includes a single secondary cannabinoid. In another embodiment, the secondary cannabinoid profile comprises two or more secondary cannabinoid. As used herein, “to match a secondary cannabinoid profile” means in one embodiment to provide in the cannabis placebo composition the same secondary cannabinoids at substantially the same quantity as in the active cannabis product. In another embodiment, “to match a secondary cannabinoid profile” means that different, but complementary secondary cannabinoid, may be used. By “complementary” it is meant that individually or in sum the secondary cannabinoid provide substantially the effect (e.g. psychoactive effect) as provided by the secondary cannabinoids of the active cannabis product.

As mentioned above, these types of placebo compositions that comprise secondary cannabinoids are advantageous for studying the effect of active agents in combination with cannabinoids.

Combination products may be beneficial for a number of reasons. For one, combinations of cannabinoids with other active agents, including other cannabinoids and/or non-cannabinoid therapeutic drugs may provide beneficial treatments for diseases and disorders. In particular, both THC and CBD have been associated with beneficial therapeutic properties, and combining these therapeutic properties with those of other active agents may be particularly beneficial. The cannabis placebo composition of the present disclosure will allow for single- or double-blind clinical trials to be conducted by providing placebos that are indistinguishable from the active cannabis product.

Exemplary therapeutic and/or recreational benefits of THC include, without limitation, reduced risk of cardiovascular disease, inhibition of tumor growth (e.g. glioblastoma), anti-tumor properties in respect of breast cancer, anti-inflammatory properties, anticonvulsant properties, stimulates appetite, reduces nausea, assists in pain management, reduces symptoms of multiple sclerosis, promotes brain growth, enhances senses, acts as an antibacterial, acts as an antioxidant, acts as a bronchodilator, increases deep sleep and prevention of insomnia, prevention of seizures, increased insulin sensitivity, and reduces amyloid plaque levels.

Exemplary therapeutic and/or recreational benefits of CBD include, without limitation, pain relief, reduces anxiety and depression, alleviates cancer-related symptoms, reduces acne, has neuroprotective properties, benefits heart health, antipsychotic effects, aids in substance abuse treatment, and diabetes prevention.

Thus, combining THC and/or CBD with any number of active agents that likewise treat or ameliorate these symptoms or the underlying disease or disorder may represent an advantageous research initiative, a number of which are currently underway. The cannabis placebo composition of the present disclosure will allow for single- or double-blind clinical trials to be conducted by providing placebos that are indistinguishable from the respective active cannabis products (i.e. combination products).

In addition, both THC and CBD have been associated with adverse side effects when used for other purposes, be it therapeutic or recreational. For example, THC has been found to cause schizophrenia-like symptoms, altered perception, increased anxiety, delayed recall time, impaired mental performance, increased cortisol levels, impairment of working memory, reduced dopamine levels, impaired creativity, and divergent thinking, just to name a few. While many of these properties may be desired for recreational use of THC, those who require THC for therapeutic purposes may prefer to avoid these adverse side effects. CBD ha been found to cause diarrhea, changes in appetite and fatigue.

In an embodiment, the cannabis placebo composition of the present disclosure comprises one or more one or more secondary cannabinoids that are psychoactive cannabinoids. In an embodiment, the cannabis placebo composition comprises THC as a secondary cannabinoid and may be used as a placebo for an active cannabis product that contains an active agent that counteracts an adverse effect of THC. In an embodiment, the active agent is an appetite suppressant, an antipsychotic, an anti-anxiety drug, or a nootropic. Any appetite suppressant, antipsychotic, anti-anxiety drug, nootropic, or combination thereof may be used. The identity and availability of specific appetite suppressant, antipsychotic, anti-anxiety drug, and nootropic active agents (e.g. drugs) would be well-known to the skilled person.

In an embodiment, the cannabis placebo composition of the present disclosure comprises one or more one or more secondary cannabinoids that are non-psychoactive cannabinoids. In an embodiment, the cannabis placebo composition comprises CBD as a secondary cannabinoid and may be used as a placebo for an active cannabis product that contains an active agent that counteracts an adverse effect of CBD. In an embodiment, the active agent is an anti-diuretic, an appetite suppressant, an appetite stimulant, or an energy-boosting substance. Any anti-diuretic, appetite suppressant, appetite stimulant, energy-boosting substance, or combination thereof may be used. The identity and availability of specific anti-diuretic, appetite suppressant, appetite stimulant, and energy-boosting active agents (e.g. drugs) would be well-known to the skilled person.

As the skilled person will appreciate, cannabis placebo compositions of the present disclosure can be purposefully designed to provide a placebo that is indistinguishable from an active cannabis product in respect of appearance (e.g. colour), odor and flavour, as well as, in certain embodiments, in respect of observable effects that are not under study.

### Methods

The present disclosure provides methods for colour matching a cannabis placebo composition to a corresponding active cannabis product, and methods for colour neutralization of cannabis products, including cannabis placebo compositions and active cannabis products.

In an embodiment, the method of producing a colour-matched placebo comprises: (i) providing an active sample of an active cannabis product; (ii) analyzing the active sample by spectrophotometry to obtain one or more absorbance measurements between 380 nm to 780 nm; (iii) converting the one or more absorbance measurements into absolute CIE XYZ values; and (iv) adding one or more colourants to a cannabis placebo composition to match the absolute CIE XYZ values of the active sample, thereby producing a colour-matched placebo.

By “active sample”, it is meant a sample of an active cannabis product. The active cannabis product is as described herein and comprises one or more active agents that are not present in a corresponding cannabis placebo composition. Active agents are described herein and may be a cannabinoid, a non-cannabinoid active agent (e.g. a drug), or any combination thereof.

The active sample is analyzed by spectrometry to obtain the absorbance value at one or more wavelengths between 380 nm and 780 nm. In an embodiment, a visible spectrophotometer may be used to obtain the absorbance measurements. A visible spectrophotometer typically measures in the visible region of the electromagnetic spectrum (340-750 nm), although Biochrom spectrophotometers can measure up to 1100 nm (the near infrared region). More preferably, a UV-Visible (UV-Vis) spectrophotometer is used in the methods disclosed herein. UV-Vis spectroscopy offers increased flexibility and is suitable for applications in the wavelength range 190 to 1100 nm. Another name for a UV-Vis spectrophotometer is a UV-Vis spectrometer.

Any number of absorbance measurements may be taken in the practice of the methods herein. In an embodiment, an absorbance measurement is collected at every 25 nm from 380 nm to 780 nm (e.g. 380 nm, 405 nm, 430 nm and so on to 780 nm inclusive). In an embodiment, an absorbance measurement is collected at every 10 nm from 380 nm to 780 nm (e.g. 380 nm, 390 nm, 400 nm and so on to 780 nm inclusive). In a preferred embodiment, an absorbance measurement is collected at every 5 nm from 380 nm to 780 nm (e.g. 380 nm, 385 nm, 390 nm and so on to 780 nm inclusive). Thus, in an embodiment, a UV-Vis spectrometer is setup and performs a scan from 780 nm to 380 nm collecting an absorbance measurement every 5 nm.

In the practice of the methods herein, the step of analyzing the active sample by spectrophotometry to obtain absorbance measurements between 380 nm to 780 nm may comprise the following steps:
1. Dilute the active cannabis product until the highest absorbance value measured on the spectrophotometer is 1 or less.
2. Setup the UV-Vis spectrophotometer to perform a scan from 780 nm to 380 nm collecting an absorbance measurement, e.g., at every 5 nm.
3. Zero the instrument using a blank sample, then perform the scan of the active cannabis product as described in step 2.
4. Collect the absorbance values from the scan.

The collected absorbance values are then converted into absolute CIE XYZ values using a CIE XYZ calculator. A CIE XYZ calculator for use in the methods of the present invention is depicted below.

TABLE 1
CIE XYZ calculator

| | Absorbance | Transmittance | | | |
|---|---|---|---|---|---|
| | (Experi- | (Calculated by | | | |
| | mentally | T = 10\hat{ }(2 − A)/ | | | |
| nm | Measured) | 100) | CIE X | CIE Y | CIE Z |
| 780 | | | 0.000042 | 0.000015 | 0 |
| 775 | | | 0.000059 | 0.000021 | 0 |
| 770 | | | 0.000083 | 0.00003 | 0 |
| 765 | | | 0.000117 | 0.000042 | 0 |
| 760 | | | 0.000166 | 0.00006 | 0 |
| 755 | | | 0.000235 | 0.000085 | 0 |
| 750 | | | 0.000332 | 0.00012 | 0 |
| 745 | | | 0.000476 | 0.000172 | 0 |
| 740 | | | 0.00069 | 0.000249 | 0 |
| 735 | | | 0.001 | 0.000361 | 0 |
| 730 | | | 0.00144 | 0.00052 | 0 |
| 725 | | | 0.002049 | 0.00074 | 0 |
| 720 | | | 0.002899 | 0.001047 | 0 |
| 715 | | | 0.004109 | 0.001484 | 0 |
| 710 | | | 0.00579 | 0.002091 | 0 |
| 705 | | | 0.008111 | 0.002929 | 0 |
| 700 | | | 0.011359 | 0.004102 | 0 |
| 695 | | | 0.01584 | 0.005723 | 0 |
| 690 | | | 0.0227 | 0.00821 | 0 |
| 685 | | | 0.0329 | 0.01192 | 0 |
| 680 | | | 0.04677 | 0.017 | 0 |
| 675 | | | 0.0636 | 0.0232 | 0 |
| 670 | | | 0.0874 | 0.032 | 0 |
| 665 | | | 0.1212 | 0.04458 | 0 |
| 660 | | | 0.01649 | 0.061 | 0 |
| 655 | | | 0.2187 | 0.0816 | 0 |
| 650 | | | 0.2835 | 0.107 | 0 |
| 645 | | | 0.3608 | 0.1382 | 0.00001 |
| 640 | | | 0.4479 | 0.175 | 0.00002 |
| 635 | | | 0.5419 | 0.217 | 0.00003 |
| 630 | | | 0.6424 | 0.265 | 0.00005 |
| 625 | | | 0.7514 | 0.321 | 0.0001 |
| 620 | | | 0.85445 | 0.381 | 0.00019 |
| 615 | | | 0.9384 | 0.4412 | 0.00024 |
| 610 | | | 1.0026 | 0.503 | 0.00034 |
| 605 | | | 1.0456 | 0.5668 | 0.0006 |
| 600 | | | 1.0622 | 0.631 | 0.0008 |
| 595 | | | 1.0567 | 0.6949 | 0.001 |
| 590 | | | 1.0263 | 0.757 | 0.0011 |
| 585 | | | 0.9786 | 0.8163 | 0.0014 |
| 580 | | | 0.9163 | 0.87 | 0.00165 |
| 575 | | | 0.8425 | 0.9154 | 0.0018 |
| 570 | | | 0.7621 | 0.952 | 0.0021 |
| 565 | | | 0.6784 | 0.9786 | 0.00275 |
| 560 | | | 0.5945 | 0.995 | 0.0039 |
| 555 | | | 0.51205 | 1 | 0.00575 |
| 550 | | | 0.43345 | 0.99495 | 0.00875 |
| 545 | | | 0.3597 | 0.9803 | 0.0134 |
| 540 | | | 0.2904 | 0.954 | 0.0203 |
| 535 | | | 0.22575 | 0.91485 | 0.02984 |
| 530 | | | 0.1655 | 0.862 | 0.04216 |
| 525 | | | 0.1096 | 0.7932 | 0.05725 |
| 520 | | | 0.06327 | 0.71 | 0.07825 |
| 515 | | | 0.0291 | 0.6082 | 0.1117 |
| 510 | | | 0.0093 | 0.503 | 0.1582 |
| 505 | | | 0.0024 | 0.4073 | 0.2123 |
| 500 | | | 0.0049 | 0.323 | 0.272 |
| 495 | | | 0.0147 | 0.2586 | 0.3533 |
| 490 | | | 0.03201 | 0.20802 | 0.46518 |
| 485 | | | 0.05795 | 0.1693 | 0.6162 |
| 480 | | | 0.09564 | 0.13902 | 0.81295 |
| 475 | | | 0.1421 | 0.1126 | 1.0419 |
| 470 | | | 0.19536 | 0.09098 | 1.28764 |
| 465 | | | 0.2511 | 0.0739 | 1.5281 |
| 460 | | | 0.2908 | 0.06 | 1.6692 |
| 455 | | | 0.3187 | 0.048 | 1.7441 |
| 450 | | | 0.3362 | 0.038 | 1.77211 |
| 445 | | | 0.34806 | 0.0298 | 1.7826 |
| 440 | | | 0.34828 | 0.023 | 1.74706 |
| 435 | | | 0.3285 | 0.01684 | 1.62296 |
| 430 | | | 0.2839 | 0.0116 | 1.3856 |
| 425 | | | 0.21477 | 0.0073 | 1.03905 |
| 420 | | | 0.13438 | 0.004 | 0.6456 |
| 415 | | | 0.07763 | 0.00218 | 0.3713 |
| 410 | | | 0.04351 | 0.00121 | 0.2074 |
| 405 | | | 0.02319 | 0.00064 | 0.1102 |
| 400 | | | 0.01431 | 0.000396 | 0.06785 |
| 395 | | | 0.00765 | 0.000217 | 0.03621 |
| 390 | | | 0.004243 | 0.00012 | 0.02005 |
| 385 | | | 0.002236 | 0.000064 | 0.01055 |
| 380 | | | 0.001368 | 0.000039 | 0.00645 |

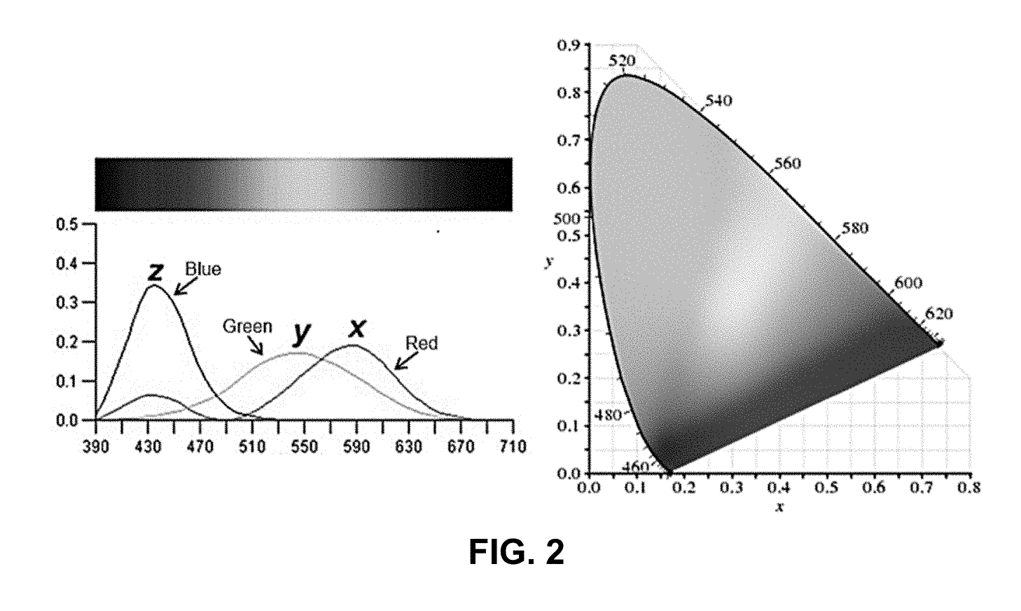

CIE 1931 XYZ is a 3-dimensional representation of perceived colour, it provides an X-curve, Y-curve, and Z-curve (see FIG. 2 for CIE 1931 XYZ color space diagrams; colour versions available on Wkipedia under “CEI 1931 Color Space”). These three curves correspond to the three types of cones present in the human eye. Depending on the colour observed, different cones are activated at different proportions and intensities.

Generally, to perform the colour match of the present disclosure, a sample of the active cannabis product is collected and analyzed for absorbance on a spectrophotometer as described above, followed by the CIE 1931 XYZ colour quantification method described below. A sample of a corresponding cannabis placebo composition is likewise analyzed by the same methods. Once CIE XYZ values have been generated from the CIE XYZ calculator for the active cannabis product and the cannabis placebo composition, the CIE XYZ values may be compared and one or more colourants may be added to the cannabis placebo composition to match the CIE XYZ values of the active cannabis product, thereby producing a colour-matched placebo. This process is described in greater detail below and may be supplemented by common general knowledge of CIE 1931 XYZ colour matching functions.

Having obtained the absorbance measurements as described above, the CIE XYZ calculator takes the absorbance values from the analysis, and converts them to transmittance values using the equation T=10\hat{ }(2−A)/100, where T is transmittance and A is absorbance. These transmittance values are corrected using the CIE XYZ functions (i.e. by converting each transmittance value to corrected CIE X, CIE Y and CIE Z values in accordance with the correction factor for each of X, Y and Z in Table 1), and the corrected CIE X, CIE Y and CIE Z values are summed for each measurement between 780 nm and 380 nm to calculate the relative CIE values (relative CIE X, CIE Y and CIE Z). Using maximum possible values for CIE XYZ (i.e. max value for X, Y and Z), the relative values are corrected to generate absolute CIE XYZ values (i.e. absolute X, Y and Z values). In this regard, as used herein, the maximum possible value for X=0.9642, Y=1, and Z=0.8252.

This same process can be performed on both the active cannabis product and the cannabis placebo composition.

The difference between the absolute CIE X, Y, and Z, values of the active cannabis product and the cannabis placebo product represents the difference in perceived colour of the two samples. The CIE X value represents the Red wavelength, the CIE Y value represents the Green wavelength and the CIE Z value represents the Blue wavelength (see FIG. 2).

To colour match a cannabis placebo composition to an active cannabis product, the difference in CIE XYZ values between the cannabis placebo composition (to be matched) and the active cannabis product are compared. The differences in absolute CIE XYZ values will be corrected by adding colourants to the cannabis placebo composition. The colourant used in the methods herein may be any colourant as described herein. Addition of these colourants is used to modify the CIE XYZ values of the cannabis placebo composition to match the colour of the active cannabis product.

In an embodiment, the difference in CIE XYZ values between the cannabis placebo composition and the active cannabis product are resolved by using colourants whose effect on the CIE XYZ values have been experimentally determined, allowing for specific manipulations of perceived colour.

In an embodiment, to experimentally calculate the effect of a colourant on CIE XYZ values:
First, analysis and quantification of colour should be performed on the base components (e.g. MCT oil) by the methods described herein to obtain the absorbance measurements between 380 nm and 780 nm (e.g. steps 1-4, above).
Second, the absorbance measurements should be converted into absolute CIE XYZ values. This generates the CIE XYZ values of the blank base component prior to the addition of any colourant.
Third, a known quantity of colourant is added to the base component, and steps 1-4 are repeated.
Fourth, the absorbance measurements should be converted into absolute CIE XYZ values. This generates the CIE XYZ values. Notably, depending on the colour of the colourant being used, changes may only be observed on one of the X, Y, or Z values.
Fifth, the process is repeated 3-5 times using a different quantity (e.g. increasing quantity) of colourant each time. This allows for the creation of a table effectively outlining the effect of the colourant on the CIE XYZ values as an increasing quantity of colourant is added to the base component.

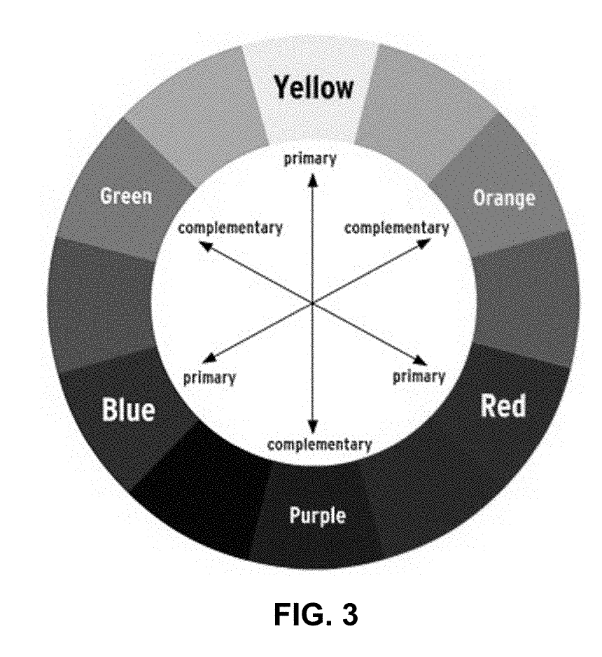

The calculated value for each colourant describes how the absolute CIE XYZ values are affected by the quantity of colourant added, this quantity is measured in percent weight by weight (e.g.: +0.05 absolute CIE X/1% (w/w) colourant added). By utilizing properties of colour theory (see FIGS. 2 and 3), combined with experimentally calculated colourant effect values, colour-match placebos can be produced that are identical in terms of colour, flavour and odor to active cannabis products.

With respect to flavour and odor, the methods herein may additionally comprise adding terpenes, esters, flavonoids, or any combination thereof to the cannabis placebo composition to match the flavour and odor of the active cannabis product. In an embodiment, the flavour and odor are matched before the colour matching methods are performed. In this manner, it is ensured that if the terpenes, esters or flavonoids have any impact on colour, it is adjusted for by the colour matching methods being performed after their addition.

In an embodiment, the terpenes used for flavour and odor matching are cannabis-derived terpenes. The terpenes extracted from dried cannabis flower produce an authentic cannabis flavour without the presence of psychoactive cannabinoids. Terpene concentrations in cannabis flower are inherently variable, therefore to create a cannabis placebo composition that is representative of an active cannabis product in terms of flavour and odor, the terpenes added should be present in a quantity that is representative of the product to be matched. In essence, this involves a matching of the terpene profile found in the active cannabis product, as described elsewhere herein.

Taking into account the foregoing, in an embodiment the methods herein for producing a colour-matched placebo comprise:
(i) providing an active sample of an active cannabis product and a placebo sample of a cannabis placebo product;
(ii) analyzing the active sample and the placebo sample by spectrometry to obtain one or more absorbance measurements between 380 nm to 780 nm for each of the active sample and the placebo sample, preferably with absorbance measurements being taken every 5 nm;
(iii) converting the absorbance measurements for each of the active sample and the placebo sample to transmittance values;
(iv) correcting the transmittance values using the CIE XYZ functions as shown in Table 1;
(v) obtaining the sum of the corrected CIE X values between 380 nm to 780 nm, the sum of the corrected CIE Y values between 380 nm to 780 nm, and the sum of the corrected CIE Z values between 380 nm to 780 nm, to calculate the relative CIE XYZ vales;
(vi) correcting the relative CIE XYZ values using the maximum possible values for CIE XYZ to generate the absolute CIE XYZ values for each of the placebo sample and the active sample;
(vii) determining the difference between the absolute CIE XYZ values of the placebo sample and the active sample;
(viii) adding one or more colourants to the cannabis placebo composition to match the absolute CIE XYZ values to that of the active cannabis product, preferably the effect of the colourant having been experimentally determined as described herein;
(ix) if necessary, repeating the process of steps (i) to (vili) until a colour-matched placebo is obtained, if it was not already obtained.

As the skilled person will appreciate, the steps of the method described herein for producing a colour-matched placebo can be performed in various different order. For example, in an embodiment the absolute CIE XYZ values of the active cannabis product and the cannabis placebo composition are determined side-by-side (e.g. concurrently). In another embodiment, the absolute CIE XYZ values of the active cannabis product are determined first, followed by the absolute CIE XYZ values of the cannabis placebo composition. In another embodiment, the absolute CIE XYZ values of the cannabis placebo composition are determined first, followed by the absolute CIE XYZ values of the active cannabis product. Many further alternatives would be readily apparent to the skilled person in the art.

The present disclosure further provides methods for colour neutralizing the colour of cannabis products, including cannabis placebo compositions and active cannabis products.

In an embodiment, the method of neutralizing the colour of a cannabis product comprises: (i) providing a sample of a cannabis placebo composition or an active cannabis product; (ii) analyzing the sample by spectrophotometry to obtain one or more absorbance measurements between 380 nm to 780 nm; (iii) converting the one or more absorbance measurements into an absolute CIE XYZ value; and (iv) adding a colourant that is opposite to an undesired colour to reduce the effect of the undesired colour, thereby neutralizing the colour of the cannabis placebo composition or an active cannabis product.

In the methods of neutralizing the colour of a cannabis product, steps (i) to (iii) may be performed as described herein in respect of the methods of producing a colour-matched placebo. By “cannabis product”, it is meant any cannabis placebo composition or active cannabis product as described herein.

As opposed to the colour matching methods described herein, when neutralizing the colour of a product the CIE XYZ values need to be corrected in the opposite direction. To neutralize any specific colour, a colour that is opposite on the colour wheel (see FIG. 3) is added to neutralize the undesired colour (i.e. unwanted wavelengths). Similar to the methods for producing a colour-matched placebo, the effect of colourants on absolute CIE XYZ values can be determined to assist in identifying the appropriate opposing colour and determining the amount that should be added to provide the desired colour neutralization.

In an embodiment of any of the methods described herein, the cannabis placebo composition is an oil. In such embodiments, the active cannabis product is a corresponding cannabis-containing oil.

In an embodiment of any of the methods described herein, the cannabis placebo composition is a gel capsule.

In an embodiment of any of the methods described herein, the cannabis placebo composition is a liquid preparation, such as a beverage.

In a further aspect of the present disclosure, there is provided a cannabis placebo composition prepared by the methods as described herein for producing a colour-matched placebo and/or neutralizing the colour of a cannabis product.

The methods disclosed herein provide various advantages. For one, the methods of colour matching and colour neutralization mitigate variance between cannabis placebo compositions and active cannabis products by reducing the effect of human perception. The colours are matched and/or neutralized experimentally using calculated CIE XYZ values, rather than based on human perception which can vary between individual. For another, the methods of colour matching and colour neutralization represent significant time-saving methods, particularly after the experimental dye effect values have been determined for particular colourants for cannabis products. For another, the creation herein of the CIE XYZ calculator saves money by eliminating the need to purchase expensive software.

### EXAMPLES

### Example 1

A cannabis placebo composition of the present disclosure was prepared.

Using medium chain triglyceride (MCT) oil, Beta-Carotene extract, Alfalfa extract, and cannabis-derived terpenes, a naturally derived cannabis placebo oil was prepared that is identical and indistinguishable from cannabis oil in terms of flavour, smell and colour.

By the methods disclosed herein, the cannabis placebo oil can be colour matched to active cannabis products. The precise amounts of the exemplified components may vary based on cannabis strain, type of oil, batch, lot, etc. In this regard, the methods herein can be used to determine the exact quantity to be used to prepare a colour-matched placebo for each respective active cannabis product.

The cannabis placebo composition (oil) of this example was compared to three different active cannabis oil products that are commercially available, namely Spectrum Red Cannabis Oil (sativa), Argyle Cannabis Oil (indica) and Spectrum Yellow Cannabis Oil (hybrid).

Spectrum Red Cannabis Oil is a product that comprises about 26.3 mg/mL THC and <1 mg/mL CBD, and has a terpene profile comprising Beta-Caryophyllene (about 40-55%), Pinene (about 25-35%), Myrcene (about 10-20%), Limonene (about 1-10%) and Terpineol (about 1-10%).

Argyle Cannabis Oil is a product that comprises about 2.5 mg/spray THC and about 3.0 mg/spray CBD, and has a terpene profile comprising Myrcene (about 35-45%), Pinene (about 25-35%), Caryophyllene (about 15-25%), and Limonene (about 5-10%).

Spectrum Yellow Cannabis Oil is a product that comprises <1 mg/mL THC and about 20.0 mg/mL CBD, and has a terpene profile comprising Myrcene (about 45-55%), Beta-Caryophyllene (about 35-45%), Pinene (about 1-10%), and Terpineol (about 1-10%).

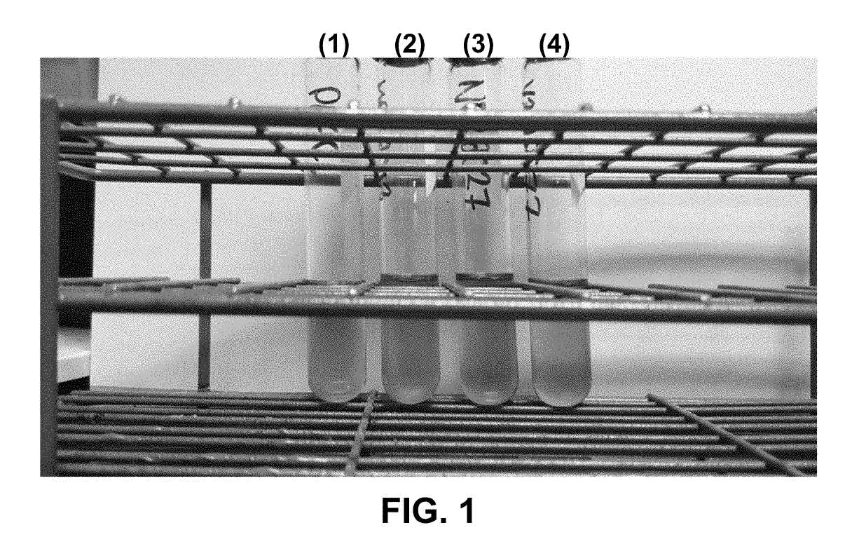

As shown in FIG. 1, it was possible to prepare a cannabis placebo oil that was colour matched to these three active commercial products. In FIG. 1, from left to right: (1) Placebo Cannabis Oil, (2) Spectrum Red Cannabis Oil, (3) Argyle Oil, and (4) Spectrum Yellow Cannabis Oil. Although it cannot be observed in the black-and-white rendering, the compositions had a near identical honey colour (yellowish-brown).

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. A cannabis placebo composition comprising:
a base component matched to that of an active cannabis product;
a colourant to provide a colour match between the cannabis placebo composition and the active cannabis product; and
a terpene, an ester, a flavonoid, or any combination thereof to provide an odor and flavour match between the cannabis placebo composition and the active cannabis product,
wherein the cannabis placebo composition is indistinguishable in appearance, odor and flavour from the active cannabis product.

2. The cannabis placebo composition of claim 1, wherein the base component is a carrier oil.

3. The cannabis placebo composition of claim 2, wherein the carrier oil is medium chain triglyceride (MCT) oil, long chain triglyceride (LCT) oil, coconut oil, corn oil, canola oil, olive oil, avocado oil, vegetable oil, flaxseed oil, palm oil, peppermint oil, hemp oil, sesame oil, sunflower oil, a winterized oil of long-chain mono-, di-, and tri-glycerides, rice bran oil, or any combination thereof.

4. The cannabis placebo composition of claim 1, wherein the colourant is derived from a source other than a cannabis plant.

5. The cannabis placebo composition of claim 1, wherein the colourant is synthetically produced or is derived from an extract from a plant other than cannabis.

6. The cannabis placebo composition of claim 1, wherein the colourant is comprised of anthocyanins, beetroot, caramel, carbo black, carmine, carotenoids, chlorophyll, curcumin, paprika, riboflavin, titanium dioxide, or any combination thereof.

7. The cannabis placebo composition of claim 1, wherein the colourant is comprised of a beta-carotene extract, an alfalfa extract, or any combination thereof.

8. The cannabis placebo composition of claim 1, which comprises one or more terpenes to match a terpene profile of the active cannabis product.

9. The cannabis placebo composition of claim 8, wherein the one or more terpenes are cannabis-derived terpenes.

10. The cannabis placebo composition of claim 8, wherein the one or more terpenes are 7,8 dihydro-alpha-ionone, 7,8-dihydro-beta-ionone, Acetanisole, Acetic Acid, Acetyl Cedrene, Anethole, Anisole, Benzaldehyde, Bergamotene, Bisabolol, Borneol, Bornyl Acetate, Butanoic/Butyric Acid, Cadinene, Cafestol, Caffeic acid, Camphene, Camphor, Capsaicin, Carene, Carotene, Carvacrol, Dextro-Carvone, Laevo-Carvone, Caryophyllene, Caryophyllene oxide, Cedrene, Cedrene Epoxide, Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene, Decanal, Dehydrovomifoliol, Diallyl Disulfide, Dihydroactinidiolide, Dimethyl Disulfide, Eicosane/Icosane, Elemene, Estragole, Ethyl acetate, Ethyl Cinnamate, Ethyl maltol, Eucalyptol/1,8-Cineole, Eudesmol, Eugenol, Euphol, Farnesene, Farnesol, Fenchol, Fenchone, Geraniol, Geranyl acetate, Germacrenes, Germacrene B, Guaia-1(10),11-diene, Guaiacol, Guaiene, Gurjunene, Herniarin, Hexanaldehyde, Hexanoic Acid, Hexyl acetate Humulene, Ionol, Ionone, Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Longipinene, Lycopene, Menthol, Methyl butyrate, 3-Mercapto-2-Methylpentanal, Mercaptan/Thiols, Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene, Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Pinene, Propanethiol, Pristimerin, Pulegone, Phytol, Quercetin, Retinol, Rutin, Sabinene, Sabinene Hydrate, cis-Sabinene Hydrate, trans-Sabinene Hydrate, Safranal, Selinene, Sinensal, Sitosterol, Squalene, Taxadiene, Terpin hydrate, Terpineol, Terpine-4-ol, Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, Vanillin, or any combination thereof.

11. The cannabis placebo composition of claim 8, wherein the one or more terpenes are Beta-Myrcene, Linalool, Alpha-Pinene, Beta-Pinene, Beta-Caryophyllene, Caryophyllene oxide, Alpha-Humulene, Nerolidol, D-Limonene, L-Limonene, Para-Cymene, Eugenol, Farnesol, Geraniol, Phytol, Menthol, Terpineol, Alpha-Terpineol, Benzaldehyde, Hexyl acetate, Methyl Salicylate, Eucalyptol, Ocimene, Terpinolene, Alpha-Terpinene, Isopulegol, Guaicol, Alpha-Bisabolol, or any combination thereof.

12. The cannabis placebo composition of claim 1, which comprises one or more esters selected from Benzyl acetate, Benzyl formate, Ethyl acetate, Methyl acetylsalicylate, Methyl benzoate, or any combination thereof.

13. The cannabis placebo composition of claim 1, which comprises one or more flavonoids selected from anthocyanidins, anthoxanthins, flavanones, flavanonols, flavens, or any combination thereof.

14. The cannabis placebo composition of claim 1, which consists of only naturally derived components.

15. The cannabis placebo composition of claim 1, which comprises MCT oil, Beta-Carotene extract, Alfalfa extract and two or more cannabis-derived terpenes.

16. The cannabis placebo composition of claim 1, which consists of MCT oil, Beta-Carotene extract, Alfalfa extract and two or more cannabis-derived terpenes.

17. The cannabis placebo composition of claim 1, which is devoid of cannabinoids.

18. The cannabis placebo composition of claim 1, which comprises a controlled amount of one or more secondary cannabinoids to match a secondary cannabinoid profile of the active cannabis product, and is devoid of an active agent that is present in the active cannabis product.

19. The cannabis placebo composition of claim 18, wherein the active agent is a primary cannabinoid or a drug.

20. The cannabis placebo composition of claim 18, wherein the one or more secondary cannabinoids are psychoactive cannabinoids.

21. The cannabis placebo composition of claim 20, wherein the one or more secondary cannabinoids comprise tetrahydrocannabinol (THC) and the active agent counteracts an adverse effect of THC.

22. The cannabis placebo composition of claim 21, wherein the active agent is an appetite suppressant, an antipsychotic, an anti-anxiety drug, or a nootropic.

23. The cannabis placebo composition of claim 18, wherein the one or more secondary cannabinoids are non-psychoactive cannabinoids.

24. The cannabis placebo composition of claim 23, wherein the one or more secondary cannabinoids comprise cannabidiol (CBD) and the active agent counteracts an adverse effect of CBD.

25. The cannabis placebo composition of claim 24, wherein the active agent is an anti-diuretic, an appetite suppressant, an appetite stimulant, or an energy-boosting substance.

26. The cannabis placebo composition of claim 1, which is in a dosage form selected from an oil, a gel capsule, a tablet, a lozenge or pastille, a pill, a granule, a powder, a cream, a suppository, an enema, or a liquid preparation.

27. A method for producing a colour-matched placebo, the method comprising:
providing an active sample of an active cannabis product;
analyzing the active sample by spectrophotometry to obtain one or more absorbance measurements between 380 nm to 780 nm;
converting the one or more absorbance measurements into absolute CIE XYZ values; and
adding one or more colourants to a cannabis placebo composition to match the absolute CIE XYZ values of the active sample, thereby producing a colour-matched placebo.

28. The method of claim 27, wherein the spectrophotometry is UV-Vis spectrophotometry.

29. The method of claim 27, which further comprises:
obtaining corresponding absolute CIE XYZ values for a placebo sample of the cannabis placebo composition; and
determining the difference between the absolute CIE XYZ values of the active sample and the placebo sample.

30. The method of claim 29, which comprises repeating the method steps until the colour-matched placebo is obtained.

31. The method of claim 27, wherein the one or more absorbance measurements are at every 5 nm between 380 nm and 780 nm.

32. The method of claim 27, which further comprises diluting the active sample until the highest absorbance measurement measured by spectrophotometry is 1 or less.

33. The method of claim 27, wherein the one or more colourants are derived from a source other than a cannabis plant.

34. The method of claim 27, wherein the one or more colourants are synthetically produced or are derived from an extract from a plant other than cannabis.

35. The method of claim 27, wherein the one ore more colourants are comprised of anthocyanins, beetroot, caramel, carbo black, carmine, carotenoids, chlorophyll, curcumin, paprika, riboflavin, titanium dioxide, or any combination thereof.

36. The method claim 27, wherein the one or more colourants are comprised of a beta-carotene extract, an alfalfa extract, or any combination thereof.

37. A method for neutralizing the colour of a cannabis placebo composition or an active cannabis product, the method comprising:
providing a sample of a cannabis placebo composition or an active cannabis product;
analyzing the sample by spectrophotometry to obtain one or more absorbance measurements between 380 nm to 780 nm;
converting the one or more absorbance measurements into absolute CIE XYZ values; and
adding a colourant that is opposite to an undesired colour to reduce the effect of the undesired colour, thereby neutralizing the colour of the cannabis placebo composition or an active cannabis product.

38. The method of claim 37, wherein the spectrophotometry is UV-Vis spectrophotometry.

39. A cannabis placebo composition prepared by the method of claim 27, wherein the cannabis placebo composition is colour matched to an active cannabis product.

40. (canceled)

41. (canceled)
